(19) United States
(12) Patent Application Publication
(10) Pub. No.: US 2026/0344999 A1
(43) Pub. Date: Nov. 5, 2026

(54) SELF-CALIBRATING DEVICES, SYSTEMS, AND METHODS OF MEASURING CORNEAL TOPOGRAPHY

(71) Applicant: Vision Labs, Inc., Mill Valley, CA (US)

(72) Inventors: Nikolai Steklov, Bakersfield, CA (US); Zachary Bodnar, Las Vegas, NV (US); Jochen Kumm, Palo Alto, CA (US)

(21) Appl. No.: 19/642,349

(22) Filed: Apr. 8, 2026

Related U.S. Application Data
(63) Continuation of application No. PCT/US2024/051283, filed on Oct. 14, 2024.
(60) Provisional application No. 63/589,932, filed on Oct. 12, 2023.

(51) Int. Cl. A61B 3/107; A61B 3/14

(52) U.S. Cl. CPC A61B 3/107; A61B 3/14

(57) ABSTRACT

Provided are devices, systems, and methods configured to uniformly illuminate a biological sample with a patterned illumination source and determine a contour of the biological sample.

### CROSS-REFERENCE

This application is a continuation application of International Patent Application No. PCT/US2024/051283, filed Oct. 14, 2024, which claims priority to U.S. Provisional Application No. 63/589,932, filed Oct. 12, 2023, each of which is incorporated herein by reference in its entirety for all purposes.

### BACKGROUND

Corneal topography has become a valuable diagnostic parameter for routine eye examination, pre-operative planning and evaluation for laser refractive surgeries (e.g., LASIK and PRK), contact lens fitting, evaluation of cataract and numerous corneal diseases such as keratoconus. Commercially available corneal topography systems (i.e., keratometers) are typically complex, expensive bench top systems located at offices of corneal specialists, laser vision correction surgeons, cataract surgeons, and eye care professionals (e.g., ophthalmologists or optometrists) who frequently measure the corneal topography of their patients. Unfortunately, the relatively high cost of these systems ($8,000 to over $50,0000) and the need of specially trained medical personal preclude the wide-spread use of keratometry for healthcare professionals located in rural and resource limited areas. In such rural areas, patients in need of an eye examination to measure their corneal topography would need to travel to the nearest large town or city that may not be feasible or convenient for repeat or follow up visits if necessary. Enabling local general practitioner physicians in rural and low resource communities to screen their patient's corneal topographies would circumvent such inefficiencies and provide rapid screening and accessibility to local longitudinal monitoring of their corneal disease. Therefore, there exists an unmet need of a rapid, low-cost point-of-care system and device configured to measure and monitor corneal topography without the need of costly equipment and specialized medical personnel.

### SUMMARY

Provided herein are devices, systems and methods of their use that can measure the corneal topography of a subject's cornea at the point-of-care without the need of expensive keratometry equipment or specially trained medical personnel. In one aspect, the low-cost aspect may be achieved by the use of a common smart phone to emit, detect, and process light signals to determine the corneal topography of a subject's cornea. In another aspect, a low-cost light source may couple to a smart phone to detect and process reflected light signals. By providing low-cost, user-friendly point-of-care devices, systems, and methods to measure corneal topography a larger patient population may, for example, undergo frequent screening of corneal topography to monitor and prevent the progression of corneal diseases e.g., corneal ectasia which may encompasses post-laser assisted in situ keratomileusis (LASIK) corneal changes, pellucid marginal degeneration, keratoconus, etc., that would otherwise only seek medical care once symptoms present.

Aspects disclosed herein provide a device for corneal topography measurement, comprising: a illumination body configured to releasably couple to a mobile device, wherein the illumination body is further configured to (a) project a light pattern onto a cornea of an eye to generate a reflected light pattern and (b) aid transmission of the reflected light pattern from the cornea to an imaging device on the mobile device for generating a plurality of light signals, wherein an optical axis of the imaging device is offset from (i) a patterned region or (ii) an illumination source on the illumination body. In some embodiments, the offset between the optical axis of the imaging device and the pattern region or the illumination source on the illumination body is from about 1 mm to about 10 mm. In some embodiments, the offset between the optical axis of the imaging device and the pattern region or the illumination source on the illumination body is from about 5 to about 360 degrees. In some embodiments, the illumination body is configured to serve as a protective casing for the mobile device. In some embodiments, the illumination body light pattern comprises a plurality of lines. In some embodiments, the plurality of lines is linear. In some embodiments, the plurality of lines is circular or radial. In some embodiments, the illumination body comprises a quick release mechanism that enables the illumination body to be releasably coupled to the mobile device. In some embodiments, the plurality of lines is provided on a surface of the illumination body. In some embodiments, the illumination body is transparent or translucent. In some embodiments, the illumination body has an absorption coefficient of at least 1 cm⁻¹. In some embodiments, the plurality of lines is opaque.

Aspects disclosed herein provide a system comprising: the device previously described; and one or more processors configured to process the plurality of light signals by (i) comparing the projected light pattern to the reflected light pattern to produce a two-dimensional elevation gradient, and (ii) using the two-dimensional elevation gradient to generate a three-dimensional topographic map of the cornea. In some embodiments, the one or more processors are located on the mobile phone. In some embodiments, the one or more processors are located on a server remote from the mobile device. In some embodiments, the system further comprises the mobile device, and wherein the mobile device comprises a depth sensor configured to measure a distance from the cornea to the imaging device.

Aspects disclosed herein provide a method of measuring corneal topography, comprising: (a) providing a illumination body having a plurality of lines, rings, and/or other geometrical patterns, described elsewhere herein; (b) coupling the illumination body to a mobile device in a configuration such that an optical axis of the imaging device is offset from a patterned region on the illumination body or an illumination source on the illumination body; (c) placing the illumination body coupled to the mobile device in proximity to an eye of a subject; (d) using the illumination body and the illumination source to project a light pattern onto the cornea to generate a reflected light pattern; (e) using the imaging device on the mobile device to receive the reflected light pattern to generate a plurality of light signals; and (f) generating a topography map of the cornea based at least in part on the plurality of light signals. In some embodiments, (e) further comprises using a depth sensor on the mobile device to detect a distance between the illumination body and the eye of the subject. In some embodiments, the plurality of lines on the illumination body are linear. In some embodiments, the plurality of lines on the illumination body are circular or radial. In some embodiments (e) further comprises rotating the imaging device as the imaging device is receiving the reflected light pattern.

Aspects disclosed herein provide an device for illuminating a target, comprising: a first surface comprising a plurality of light scattering elements and a second surface comprising a plurality of illumination elements in optical communication with the plurality of light scattering elements, wherein the first surface comprises a light inlet comprising a curved surface or waveguide configured to (i) receive light emitted from a light source and (ii) direct the light to the plurality of light scattering elements, wherein the plurality of light scattering elements is configured to transmit the light to the plurality of illumination elements, and wherein the plurality of illumination elements is configured to generate an light pattern for illuminating the target. In some embodiments, the plurality of light scattering elements comprises one or more dome reflectors, scattering particles, or any combination thereof. In some embodiments, the curved surface or waveguide is configured to receive and distribute the light from the light source to the plurality of light scattering elements while reducing or minimizing a light hot spot at or near the light inlet or the light source. In some embodiments, the light hot spot corresponds to a concentration of light at or near the light inlet. In some embodiments, the second surface comprises a reflective coating. In some embodiments, the plurality of illumination elements is spatially arranged in a predetermined pattern. In some embodiments, the plurality of illumination elements comprises a linear shape. In some embodiments, the plurality of illumination elements comprises a non-linear shape. In some embodiments, the non-linear shape comprises a curve or an arc. In some embodiments, the plurality of light scattering elements comprises a two-dimensional array of light scattering elements arranged on the first surface. In some embodiments, the illumination elements comprise one or more surfaces for illuminating the target or directing light to the target. In some embodiments, the two-dimensional array comprises a linear configuration, non-linear linear configuration, or any combination thereof. In some embodiments, the device further comprises an optically transparent window in optical communication with the light source. In some embodiments, the light source comprises a light emitting diode (LED). In some embodiments, the light source is located on a mobile device. In some embodiments, the device comprises an illumination body that is configured to serve as a protective casing for a mobile device. In some embodiments, the illumination body comprises a quick release mechanism that enables the illumination body to be releasably coupled to the mobile device. In some embodiments, the light pattern generated by the plurality of illumination elements is configured to provide a uniform illumination. In some embodiments, the uniform illumination comprises an illumination of one or more regions of the target such that a first region of the target has a brightness that is within 10% of brightness of a second region of the target. In some embodiments, the uniform illumination comprises an illumination of one or more regions of the target such that a first region of the target has a brightness that is within 0% of brightness of a second region of the target. In some embodiments, the uniform illumination comprises an illumination of one or more regions of the target such that a first region of the target has a same or similar brightness as a second region of the target. In some embodiments, the target comprises a biological tissue. In some embodiments, the biological tissue is a mammalian cornea. In some embodiments, the quick release mechanism comprises a snap-fit.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a cloud base system workflow for a point-of-care device configured to measure corneal topographical and reporting such measurements to a physician remotely, in accordance with some embodiments.

FIG. 2 shows a device system workflow for a point-of-care corneal topography measurement user device configured to measure corneal topography off-line, in accordance with some embodiments.

FIGS. 3A-3B illustrate a method for determining corneal curvature along a specific axis, in accordance with some embodiments.

FIGS. 4A-4B illustrates the cross-sectional geometry utilized to estimate the elevation of the cornea at the reflection of the first source of the light pattern (FIG. 4A) and the geometry used to estimate the elevation of the cornea at the reflection of sequential sources of the light pattern, in accordance with some embodiments (FIG. 4B).

FIGS. 5A-5B illustrate an isometric perspective view (FIG. 5A) and a top perspective view (FIG. 5B) of the illumination body, in accordance with some embodiments.

FIGS. 6A-6E illustrate the various geometries and embodiments of a depth sensing feature of the illumination body, in accordance with some embodiments.

FIG. 7 illustrates a housing configured to position and/or couple the illumination body with respect to a camera sensor and a light source of an imaging device, in accordance with some embodiments.

FIG. 8 illustrates a diagram of the illumination body optically and/or

mechanically coupled with a camera sensor and/or light source of an imaging device, and the illumination body optically coupled to a corneal surface of an eye, in accordance with some embodiments.

FIG. 9 shows a flow diagram of a method of measuring corneal topography of a subject's cornea.

FIGS. 10A-10F illustrate a projected light pattern and depth sensing feature on a curved surface (e.g., a test corneal surface) at various distances between the illumination body and the subject's cornea, in accordance with some embodiments.

FIG. 11 shows an example image captured by the systems, devices, and/or methods described herein, of the reflected light pattern on a subject's cornea used to determine corneal topography, in accordance with some embodiments.

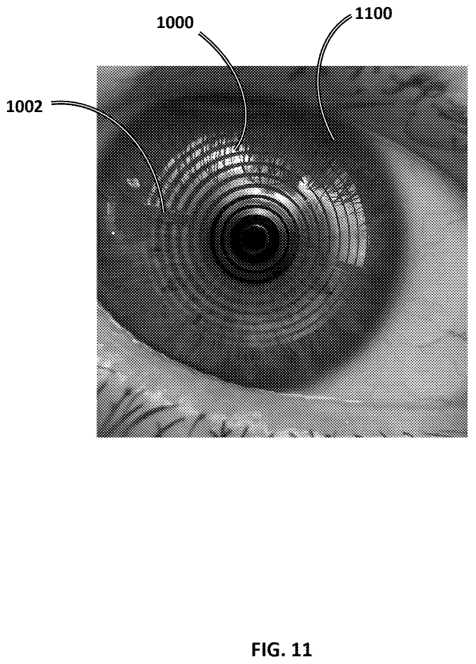

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Corneal topographic measurements can provide a rich dataset of diagnostic and prognostic factors that could greatly impact a subject's quality of life. For example, a subject interested in contact lenses can have their corneal curvature measured by keratometry to ensure proper fit and function of the contact lens. Without proper measurement of a subject's cornea and fit of a contact lens, the subject may exhibit blurry vision or pain and potential damage to the subject's eye. Additionally, corneal topographic measurements can also be used as a preventative diagnostic screening tool or to monitor corneal surgical outcomes. In some embodiments, the methods of measuring corneal topography disclosed herein may be used to determine the optimal fit or function for contact lenses. In some embodiments, the methods of measuring corneal topography disclosed herein may be used to diagnose, monitor, or screen corneal conditions, corneal diseases, pre-operative corneal structure, or post-operative changes in corneal structure. Examples of corneal conditions or disease may comprise corneal keratoconus, ectasia, pterygium formation, limbal dermoid formation or any combination thereof. In some embodiments, pre-operative corneal structure may be analyzed using the systems or methods herein, prior to using LASIK, and/or to perform LASIK corneal corrective procedures. In some embodiments, post-operative corneal structure may be analyzed using the systems or methods herein, after LASIK surgery to monitor corneal healing and the resulting change in corneal topography over time.

In some instances, the non-invasive measure of corneal topography by a costly keratometer can limit the availability and frequency by which subjects are able to monitor corneal curvature. Therefore, the present invention disclosed herein can address this shortcoming by providing systems, methods, and devices to measure corneal topography and curvature at the point-of-care.

### I. Local and Cloud-Based System

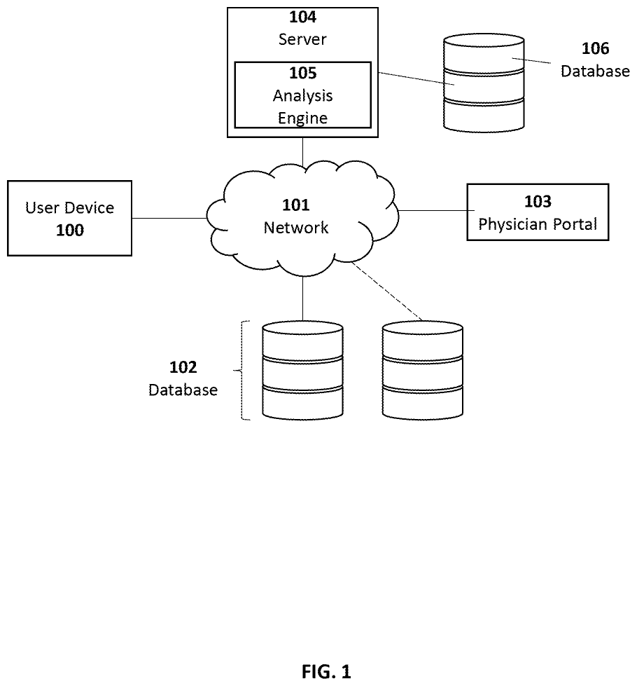

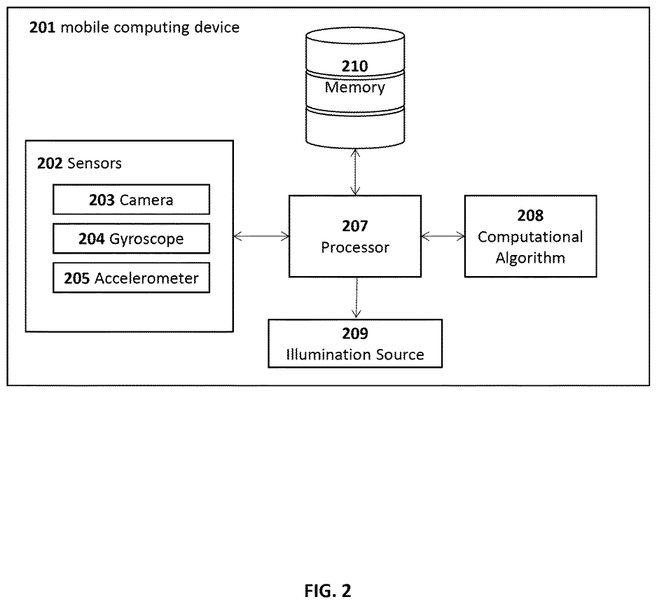

Aspects disclosed herein provide systems, methods, and/or devices, in some embodiments, that may operate locally off-line or on a cloud-based platform, as seen in FIG. 1. In some embodiments, the illumination, acquisition, image processing, and data storage may occur locally on a subject's mobile computing device 201 as seen in FIG. 2. In some embodiments, image illumination and acquisition may be completed locally on a subject's mobile computing device (100, 201) and image processing and analysis 105 may be completed on a remote server 104 through a network 101, as can be seen in FIG. 1. In some embodiments, the subject's data may also be stored in a remote HIPPA compliant database 102 that would interface with both the analysis engine 105 and a physician portal 103 through a cloud-based network 101. In some embodiments, the server 104 may comprise one or more processors that can process a subject's corneal reflected light pattern images. In some embodiments, medical personnel may remotely access a subject's corneal topographical data remotely through the physician portal 103. In some embodiments, the physician portal 103 may permit medical personnel to send recommendations and communications after viewing patient corneal data. In some embodiments, the medical personnel may complete further analysis on a subject's corneal reflected light pattern images on demand using the analysis engine 105 remotely through the network 101. In some embodiments, the analysis engine 105 may comprise a plurality of processing algorithms. In some embodiments, the plurality of processing algorithms may comprise processes for denoising, segmenting, deblurring, distortion correction, correction of chromatic aberration and extracting corneal topography from a plurality of corneal reflected light pattern images. In some embodiments, analysis engine 105, server 104 and database 106 may provide longitudinal processing and monitoring of a subject's corneal topographical data over time. In some embodiments, the longitudinal monitoring may be presented to medical personnel in an intuitive graphical user interface on the physician portal 103. In some embodiments, the cloud-based system shown in FIG. 1 may be integrated with pre-existing electronic medical record systems (EMR).

In some embodiments, the mobile computing device 201, as seen in FIG. 2, may comprise an onboard system configured to illuminate, measure, orient, process and/or store a subject's corneal topographic measurements. In some embodiments, the onboard system may comprise one or more processors 207 in electrical communication with a plurality of sensors 202, onboard memory 210, computational algorithm 208, illumination source 209, or any combination thereof. In some embodiments, the plurality of sensors 202 may comprise a camera 203, gyroscope 204, accelerometer 205, or any combination thereof sensors. In some embodiments, the camera may be a CMOS visible light camera sensitive to radiative energy in the visible spectrum. In some embodiments, the camera 203 may be a CCD visible light camera sensitive to radiative energy in the visible spectrum. In some embodiments, the camera 203 may be sensitive to radiative energy in the near-infrared spectrum. In some embodiments, the computational algorithm 208 may comprise algorithms to segment portions of captured image data, filtering and de-noising image data, measure size parameters of the segmented objects or any combination thereof. In some embodiments, the size parameters measured may comprise diameter, area, length, width, distance, circumference, or any combination thereof size parameters. In some embodiments, the segmentation algorithms may comprise manual segmentation, ellipse and/or line segmentation, Hough transform, color segmentation, or any combination thereof segmentation algorithms. In some embodiments, the segmentation algorithm may segment regions of captured image data by similar local pixel neighborhood gradients. In some embodiments, the local pixel neighborhood gradients may be aligned with one another to generate segments of smooth pixel clusters. In some embodiments, neighboring smooth pixel clusters may be combined together to generate closed contour segments. In some embodiments, the computational algorithm 208 may comprise algorithms to calculate elevation using methods described elsewhere herein of a reflected light pattern off of a subject's cornea. In some embodiments, the computational algorithm 208 may comprise image processing functions of denoising, blurring, smoothing, binarization, or any combination thereof image processing functions. In some embodiments, the computational algorithms may measure the length, width, distance, or any combination thereof the reflected light pattern from the cornea to measure a given topography at a given meridian. In some embodiments, the computational algorithm 208 may comprise a highly parallel computational structure executed on one or more parallel processors 207. In some embodiments, the highly parallel computational structure may comprise a plurality of processing cores that each may comprise a plurality of processing threads. In some embodiments, a first processing thread of a plurality of processing threads may be configured to execute a function simultaneously with a second processing thread of a plurality of processing threads. In some embodiments, a processing thread may perform a process, for example, image acquisition, pattern illumination, image segmentation, image processing, or any combination thereof processes. In some embodiments, the computational algorithm may comprise a highly parallel computation structure executed on a graphical processing unit (GPU). In some embodiments, a subject's corneal topographic information may be stored locally in memory 210 until further processing or sharing with clinical personnel. In some embodiments, a subject's corneal topographic information may be transmitted via the network 101 and stored in a cloud database 102. In some embodiments, the memory 210 may be encrypted to store personal health information (PHI) compliant under the health insurance portability and accountability act (HIPPA). In some embodiments, the PHI compliant under HIPPA may be uploaded to an electronic medical record system (EMR) for further review by clinical personnel.

### II. Measurement of Corneal Diameter and Topography

Systems, methods, and/or devices, are described herein for calculating a subject's corneal diameter and topography. Aspects of the disclosure, in some embodiments, may provide a method of determining a subject's corneal diameter by scale, geometric optics, or any combination thereof method. Further aspects of the disclosure may determine corneal topography by combining the measured corneal diameter in combination with a deviation of a captured reflected signal of a light pattern of known size and shape, as disclosed in PCT Application No. PCT/US2021/041105, which is hereby incorporated by reference.

In some embodiments, the diameter of the subject's cornea may be determined by geometric optics and ray tracing between the object (e.g., a subject's cornea) and the imaging system components. In some embodiments determining the subject's corneal diameter by geometric optics and ray tracing may depend on the diameter of the cornea in pixels of an imaging sensor, the resolution of the captured image in pixels/unit length, and/or the distance between the subject's corneal limbus and the imaging sensor's field of view. In some embodiments, the subject's corneal diameter in pixels may be determined by capturing an image of the subject's cornea with an imaging sensor with a defined number of pixels. In some embodiments, the imaging sensor may comprise a pixel size of about 1 μm to about 10 μm. In some embodiments, the imaging sensor may comprise a pixel size of about 1 μm to about 2 μm, about 1 μm to about 3 μm, about 1 μm to about 4 μm, about 1 μm to about 5 μm, about 1 μm to about 6 μm, about 1 μm to about 7 μm, about 1 μm to about 8 μm, about 1 μm to about 9 μm, about 1 μm to about 10 μm, about 2 μm to about 3 μm, about 2 μm to about 4 μm, about 2 μm to about 5 μm, about 2 μm to about 6 μm, about 2 μm to about 7 μm, about 2 μm to about 8 μm, about 2 μm to about 9 μm, about 2 μm to about 10 μm, about 3 μm to about 4 μm, about 3 μm to about 5 μm, about 3 μm to about 6 μm, about 3 μm to about 7 μm, about 3 μm to about 8 μm, about 3 μm to about 9 μm, about 3 μm to about 10 μm, about 4 μm to about 5 μm, about 4 μm to about 6 μm, about 4 μm to about 7 μm, about 4 μm to about 8 μm, about 4 μm to about 9 μm, about 4 μm to about 10 μm, about 5 μm to about 6 μm, about 5 μm to about 7 μm, about 5 μm to about 8 μm, about 5 μm to about 9 μm, about 5 μm to about 10 μm, about 6 μm to about 7 μm, about 6 μm to about 8 μm, about 6 μm to about 9 μm, about 6 μm to about 10 μm, about 7 μm to about 8 μm, about 7 μm to about 9 μm, about 7 μm to about 10 μm, about 8 μm to about 9 μm, about 8 μm to about 10 μm, or about 9 μm to about 10 μm. In some embodiments, the imaging sensor may comprise a pixel size of about 1 μm, about 2 μm, about 3 μm, about 4 μm, about 5 μm, about 6 μm, about 7 μm, about 8 μm, about 9 μm, or about 10 μm. In some embodiments, the imaging sensor may comprise a pixel size of at least about 1 μm, about 2 μm, about 3 μm, about 4 μm, about 5 μm, about 6 μm, about 7 μm, about 8 μm, or about 9 μm. In some embodiments, the imaging sensor may comprise a pixel size of at most about 2 μm, about 3 μm, about 4 μm, about 5 μm, about 6 μm, about 7 μm, about 8 μm, about 9 μm, or about 10 μm.

In some embodiments, the corneal diameter in pixels may be extracted by image processing methods of segmentation from the captured image. In some embodiments, segmentation methods may comprise segmentation by intensity thresholding, gradient thresholding, canny edge detection, Sobel edge detection, Hough transform, ellipse and line segment detector (ELSD) or any combination thereof segmentation methods. Once the shape of the cornea is segmented, the diameter of the cornea, may be determined by dividing the area of the cornea in a plurality of parallel horizontal and vertical line segments across the entirety of the segmented corneal area. In some embodiments, the average of the longest length of the vertical and horizontal line segments may indicate the diameter of the subject's cornea in pixels. In addition to the diameter of the subject's cornea in pixels, the distance between the imaging sensor and the corneal limbus may be determined to measure the diameter of the subject's cornea.

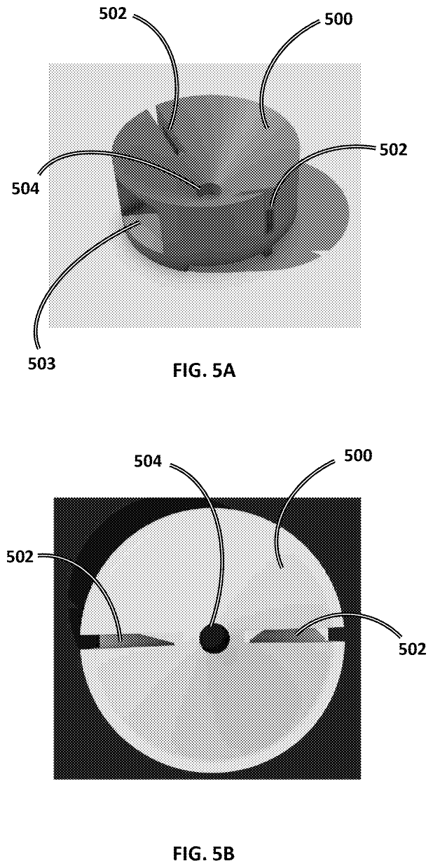

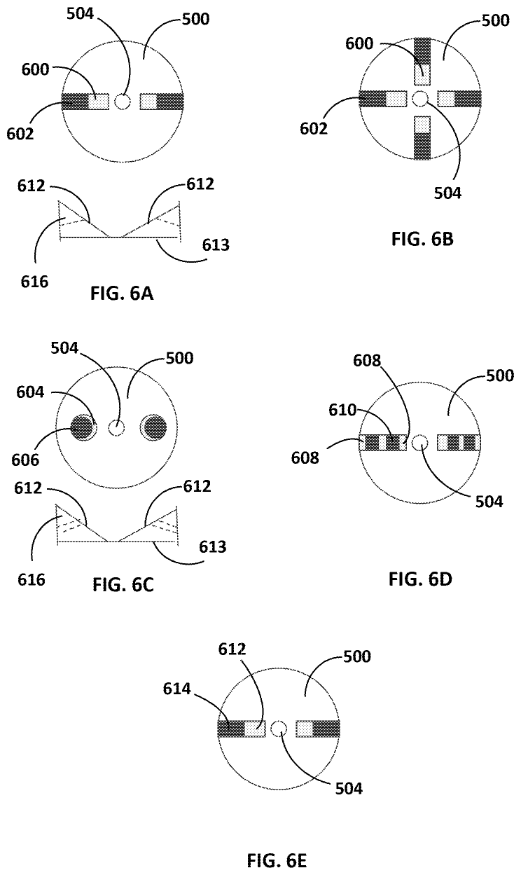

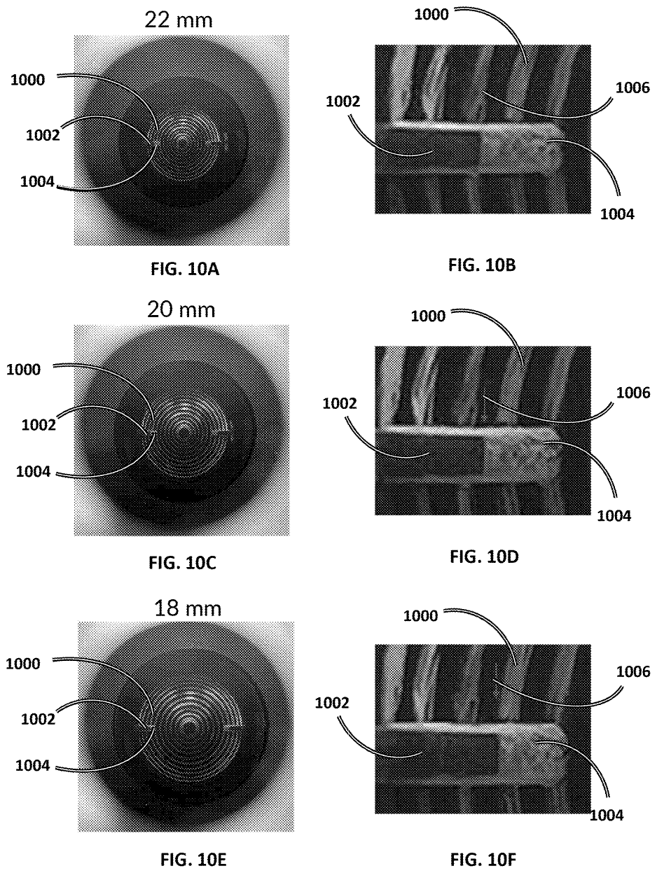

The corneal limbus is defined as the plane between the cornea (the translucent or transparent region of an eye) and the sclera (the white region of an eye). In some embodiments, the distance between the imaging sensor and the corneal limbus may be determined by a depth sensing feature (502, 600, 602, 604, 606, 608, 610, 612, 614, 616, 1002, 1004) of the illumination body 500, as shown in FIGS. 5A-5B, and FIGS. 6A-6E. In some cases, the depth sensing feature may comprise a structure that may provide the distance between the imaging sensor and the corneal limbus determined through principals of imaging perspective. For example, the position 1006, size, skew, and/or distortion of the depth sensing feature (1002, 1004) with respect to the light pattern of the topography and/or curvature sensing feature 1000 transmitted to a curved surface (e.g., a subject's cornea), as shown in FIGS. 10A-10F, may provide a distance between the imaging sensor and the corneal limbus.

In some embodiments, a geometric optics mathematical relationship combining the diameter of a subject's cornea in pixels of an imaging sensor, the imaging sensors angular field, the distance between the imaging sensor plane and the cornea limbus and the resolution of a captured image in pixels/unit length may be used to determine the diameter of the subject's cornea. For example, the diameter of the cornea may be shown by the following equation:

$$d = \frac{2 {d}^{′} z}{w} tan ( \frac{φ}{2} )$$

Where d is the diameter of the cornea,
d′ is the diameter of the cornea in pixels,
φ is the angular field of view of the camera sensor,
w is the resolution of the captured image in pixels/unit length, and
z is the distance between the camera sensor to the corneal limbus.

In some embodiments, the diameter of the cornea may be determined by a method of scale. In some embodiments, the method of determining corneal diameter by scale includes the steps of: placing a fiducial marker of known size adjacent to a subject's cornea; capturing an image of the cornea and adjacent fiducial marker; segmenting the fiducial marker from the image; determining the image pixel resolution of the image from the known scale of the fiducial marker; segmenting the cornea from the image; determining the diameter of the segmented cornea from the calculated image pixel resolution. In some embodiments, the segmentation of the fiducial marker and cornea may be achieved by segmentations methods disclosed previously herein. In some embodiments, the fiducial marker may be circular, square, rectangular, trapezoidal, triangular, ellipsoid or other regular and/or irregular polygonal shape. In some embodiments, the fiducial marker may be patterned, colored, textured or any combination thereof.

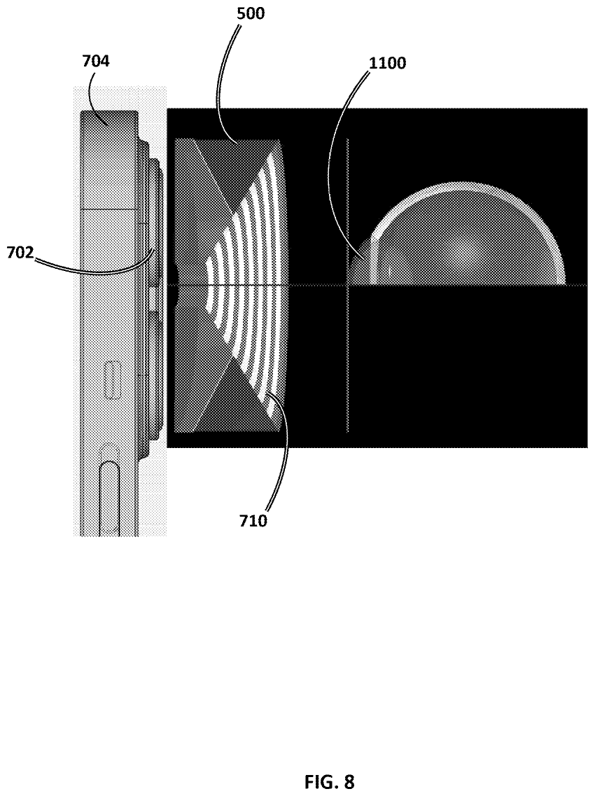

In some embodiments, corneal topography may be calculated by capturing a reflected light pattern off a subject's cornea by an imaging device. In some embodiments, the method of measuring corneal topography may comprise the steps of: providing an illumination body 500, as seen in FIGS. 5A-5B and FIG. 8, where the illumination body is configured to provide a light pattern that may comprises a depth sensing feature (502, 600, 602, 604, 606, 608, 610, 612, 614, 616), e.g., a light bar, and a topography and/or curvature sensing feature 710 (e.g., one or more linear and/or circular concentric illumination sources); coupling the illumination body 500 to an imaging device 704 (e.g., a mobile computing device camera 702) such that an optical axis of the imaging device is aligned with the optical axis of an imaging optical feature 504 (e.g., a cutout) of the illumination body 500; placing the illumination body coupled to the imaging device in proximity to an eye of a subject; projecting a light pattern (1000, 1002, 1004) of the illumination body depth sensing feature (502, 600, 602, 604, 606, 608, 610, 612, 614, 616) and the topography and/or curvature sensing feature 710, where the light pattern (1000, 1002, 1004) may comprise a plurality of lines, line segments, geometric shapes, curves, arcs, and/or concentric circular ring(s) provided to a surface of the subject's cornea 1100; detecting a reflected light pattern of the depth sensing feature (502, 600, 602, 604, 606, 608, 610, 612, 614, 616) and the topography and/or curvature sensing feature 710 off of the subject's cornea with the imaging device 704; and generating a topography map of the subject's cornea 1100 based at least in part on the reflected light pattern. In some embodiments, generating a topography map of the cornea 1100 can be achieved by a comparison of a known spacing of the light pattern (1000, 1002, 1004) and the spacing of the reflected light pattern.

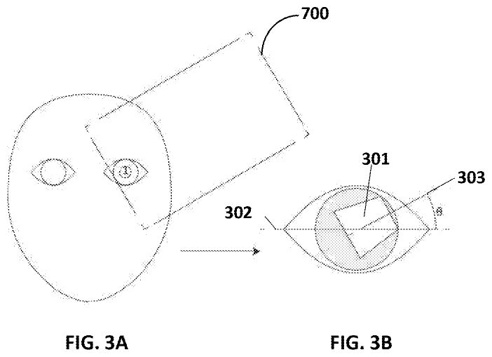

Turning to FIGS. 3A-3B, the orientation of the light pattern 301 emitted by the imaging device 704 and illumination body 500 will now be described. In some embodiments, the imaging device 704, as shown in FIG. 3A, may comprise an imaging axis that is orthogonal to a subject's corneal normal. In some embodiments, as shown in FIG. 3B, the imaging device 704 may be at an angle theta 303 with respect to the horizontal axis of the subject's cornea 302. In some embodiments, the angle theta 303 may be about 10 degrees to about 90 degrees. In some embodiments, the angle theta 303 may be about 10 degrees to about 20 degrees, about 10 degrees to about 30 degrees, about 10 degrees to about 40 degrees, about 10 degrees to about 50 degrees, about 10 degrees to about 60 degrees, about 10 degrees to about 70 degrees, about 10 degrees to about 80 degrees, about 10 degrees to about 90 degrees, about 20 degrees to about 30 degrees, about 20 degrees to about 40 degrees, about 20 degrees to about 50 degrees, about 20 degrees to about 60 degrees, about 20 degrees to about 70 degrees, about 20 degrees to about 80 degrees, about 20 degrees to about 90 degrees, about 30 degrees to about 40 degrees, about 30 degrees to about 50 degrees, about 30 degrees to about 60 degrees, about 30 degrees to about 70 degrees, about 30 degrees to about 80 degrees, about 30 degrees to about 90 degrees, about 40 degrees to about 50 degrees, about 40 degrees to about 60 degrees, about 40 degrees to about 70 degrees, about 40 degrees to about 80 degrees, about 40 degrees to about 90 degrees, about 50 degrees to about 60 degrees, about 50 degrees to about 70 degrees, about 50 degrees to about 80 degrees, about 50 degrees to about 90 degrees, about 60 degrees to about 70 degrees, about 60 degrees to about 80 degrees, about 60 degrees to about 90 degrees, about 70 degrees to about 80 degrees, about 70 degrees to about 90 degrees, or about 80 degrees to about 90 degrees. In some embodiments, the angle theta 303 may be about 10 degrees, about 20 degrees, about 30 degrees, about 40 degrees, about 50 degrees, about 60 degrees, about 70 degrees, about 80 degrees, or about 90 degrees. In some embodiments, the angle theta 303 may be at least about 10 degrees, about 20 degrees, about 30 degrees, about 40 degrees, about 50 degrees, about 60 degrees, about 70 degrees, or about 80 degrees. In some embodiments, the angle theta 303 may be at most about 20 degrees, about 30 degrees, about 40 degrees, about 50 degrees, about 60 degrees, about 70 degrees, about 80 degrees, or about 90 degrees. In some embodiments, the orientation of the imaging device 704 and illumination body 500 with respect to the cornea may be determined by onboard sensors of the mobile computing device. In some embodiments, the onboard sensors may comprise an inertial gyroscopic sensor, accelerometer, or any combination thereof sensors.

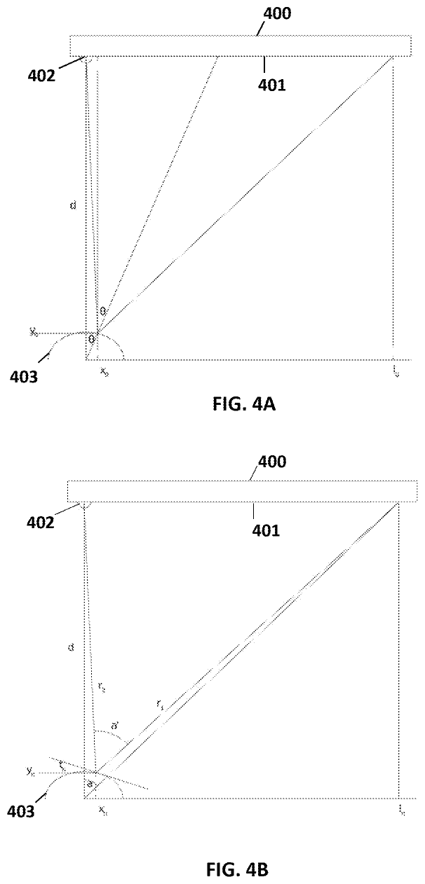

FIG. 4A-4B show the geometry and relationship between the illumination body 401, imaging device 400 (e.g., a mobile computing device), camera sensor 402 and a subject's cornea 403 utilized to calculate corneal topography. In some embodiments, an illumination body 401 may be configured to emit a light pattern onto the subject's cornea 403. In some embodiments, the light pattern may comprise parameters such as dimension, geometry, thickness, spacing, area, curvature and/or geometry of a surface of the illumination body, or any combination thereof, that are known a ‘prior. The illumination pattern reflected off of the subject's cornea 403 may then be captured by the camera sensor 402 of the imaging device 400. In some embodiments, the deviation from the known parameters of the light pattern may be used to measure corneal topography.

In some embodiments, the light pattern may comprise a series of illumination sources. In some embodiments, the illumination sources may be at an offset with respect to the camera sensor 402. In some embodiments, the offset between the optical axis of the camera sensor 402 of the imaging device 400 and the pattern region or the illumination source of the illumination body 401 may be about 1 mm to about 10 mm. In some embodiments, the offset between the optical axis of the camera sensor 402 of the imaging device 400 and the pattern region or the illumination source of the illumination body 401 may be about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 5 mm to about 6 mm, about 5 mm to about 7 mm, about 5 mm to about 8 mm, about 5 mm to about 9 mm, about 5 mm to about 10 mm, about 6 mm to about 7 mm, about 6 mm to about 8 mm, about 6 mm to about 9 mm, about 6 mm to about 10 mm, about 7 mm to about 8 mm, about 7 mm to about 9 mm, about 7 mm to about 10 mm, about 8 mm to about 9 mm, about 8 mm to about 10 mm, or about 9 mm to about 10 mm. In some embodiments, the offset between the optical axis of the camera sensor 402 of the imaging device 400 and the pattern region or the illumination source of the illumination body 401 may be about 1 mm, about 2 mm, about 3 mm, about 4 mm, about 5 mm, about 6 mm, about 7 mm, about 8 mm, about 9 mm, or about 10 mm. In some embodiments, the offset between the optical axis of the camera sensor 402 of the imaging device 400 and the pattern region or the illumination source of the illumination body 401 may be at least about 1 mm, about 2 mm, about 3 mm, about 4 mm, about 5 mm, about 6 mm, about 7 mm, about 8 mm, or about 9 mm. In some embodiments, the offset between the optical axis of the camera sensor 402 of the imaging device 400 and the pattern region or the illumination source of the illumination body 401 may be at most about 2 mm, about 3 mm, about 4 mm, about 5 mm, about 6 mm, about 7 mm, about 8 mm, about 9 mm, or about 10 mm.

In some embodiments, the offset between the optical axis of the camera sensor 402 of the imaging device 400 and the topography and/or curvature sensing feature(s) 710 of the illumination body 500 may be about 5 degrees to about 360 degrees. In some embodiments, the offset between the optical axis of the camera sensor 402 of the imaging device 400 and the topography and/or curvature sensing feature(s) 710 of the illumination body 500 may be about 5 degrees to about 40 degrees, about 5 degrees to about 80 degrees, about 5 degrees to about 120 degrees, about 5 degrees to about 160 degrees, about 5 degrees to about 200 degrees, about 5 degrees to about 240 degrees, about 5 degrees to about 280 degrees, about 5 degrees to about 320 degrees, about 5 degrees to about 360 degrees, about 40 degrees to about 80 degrees, about 40 degrees to about 120 degrees, about 40 degrees to about 160 degrees, about 40 degrees to about 200 degrees, about 40 degrees to about 240 degrees, about 40 degrees to about 280 degrees, about 40 degrees to about 320 degrees, about 40 degrees to about 360 degrees, about 80 degrees to about 120 degrees, about 80 degrees to about 160 degrees, about 80 degrees to about 200 degrees, about 80 degrees to about 240 degrees, about 80 degrees to about 280 degrees, about 80 degrees to about 320 degrees, about 80 degrees to about 360 degrees, about 120 degrees to about 160 degrees, about 120 degrees to about 200 degrees, about 120 degrees to about 240 degrees, about 120 degrees to about 280 degrees, about 120 degrees to about 320 degrees, about 120 degrees to about 360 degrees, about 160 degrees to about 200 degrees, about 160 degrees to about 240 degrees, about 160 degrees to about 280 degrees, about 160 degrees to about 320 degrees, about 160 degrees to about 360 degrees, about 200 degrees to about 240 degrees, about 200 degrees to about 280 degrees, about 200 degrees to about 320 degrees, about 200 degrees to about 360 degrees, about 240 degrees to about 280 degrees, about 240 degrees to about 320 degrees, about 240 degrees to about 360 degrees, about 280 degrees to about 320 degrees, about 280 degrees to about 360 degrees, or about 320 degrees to about 360 degrees. In some embodiments, the offset between the optical axis of the camera sensor 402 of the imaging device 400 and the topography and/or curvature sensing feature(s) 710 of the illumination body 500 may be about 5 degrees, about 40 degrees, about 80 degrees, about 120 degrees, about 160 degrees, about 200 degrees, about 240 degrees, about 280 degrees, about 320 degrees, or about 360 degrees. In some embodiments, the offset between the optical axis of the camera sensor 402 of the imaging device 400 and the topography and/or curvature sensing feature(s) 710 of the illumination body 500 may be at least about 5 degrees, about 40 degrees, about 80 degrees, about 120 degrees, about 160 degrees, about 200 degrees, about 240 degrees, about 280 degrees, or about 320 degrees. In some embodiments, the offset between the optical axis of the camera sensor 402 of the imaging device 400 and the topography and/or curvature sensing feature(s) 710 of the illumination body 500 may be at most about 40 degrees, about 80 degrees, about 120 degrees, about 160 degrees, about 200 degrees, about 240 degrees, about 280 degrees, about 320 degrees, or about 360 degrees.

In some embodiments, a first illumination source of the topography and/or curvature sensing feature(s) 710 of the illumination body 500 may be an illumination source at the shortest distance from the camera sensor 402. In some embodiments, the captured elevation of the first illumination source (y₀) may be a function of the distance of corneal reflection of the first illumination source closest to the camera sensor (x₀), the angle between the normal and the corneal reflection of the first illumination source and the y axis (θ). In some embodiments, the angle between the normal of the corneal reflection of the first illumination source and the y-axis (θ) may be a function of the distance between the first illumination source of illumination body 401 to the camera sensor (l₀), the distance between the camera sensor and the cornea apex (d), and the distance of corneal reflection of the first illumination source closest to the camera sensor (x₀) as shown in FIG. 4A. For example, the following mathematical relationship may provide the elevation of the first illumination:

$${y}_{0} = \frac{{x}_{0}}{{tan}^{- 1} ( θ )}$$

$$θ = \frac{1}{2} [ {tan}^{- 1} ( \frac{{l}_{0} - {x}_{0}}{d} ) - {tan}^{- 1} ( \frac{{l}_{0}}{d} ) ]$$

In some embodiments, each subsequent elevation (y₁, y₂, . . . yₙ) of the series of illumination sources may be computed iteratively, as shown in FIG. 4B. In some embodiments, the corneal segment between (xₙ₋₁, yₙ₋₁) and (xₙ, yₙ) may be treated as an arc segment along a circle. For example, in some embodiments, the nth subsequent elevation (yₙ) may be determined by the following mathematical relationship:

$${y}_{n} = {y}_{n - 1} - \frac{( {x}_{n - 1} - {x}_{n} ) ( cos {t}_{n - 1} - cos {t}_{n} )}{sin {t}_{n - 1} - sin {t}_{n}}$$

Where tₙ is the angle between the tangent at (xₙ, yₙ) and horizontal axis

In some embodiments, tₙ may be initially estimated to be a/2, where a is the angle between Iₙ, the nth illumination source, and the vertical axis. In some embodiments, the estimate may be refined numerically until Δyₙ=yₙ−yₙ′ stabilizes below a preset threshold. For example, the mathematical relationship that may provide an estimate for tₙ follows:

$$Δ l = {l}_{0} - {l}_{n}$$

$$Δ y = d - {y}_{n}$$

$${a}^{′} = {cos}^{- 1} \frac{{l}_{0}^{2} - 2 Δ {y}^{2} - {l}_{n}^{2} - Δ {l}^{2}}{- 2 \sqrt{( Δ {y}^{2} + {l}_{n}^{2} ) ( Δ {l}^{2} + Δ {y}^{2} )}}$$

$$t = \frac{π}{2} - \frac{{a}^{′}}{2} - {tan}^{- 1} \frac{Δ y}{Δ l}$$

$${y}_{n}^{′} = {y}_{n} - \frac{{x}_{n - 1} - {x}_{n} - ( cos {t}_{n - 1} - cos t )}{sin {t}_{n - 1} - sin t}$$

In some embodiments, the threshold may be in the range of about 0.000001 to about 0.001. In some embodiments, the threshold may be in the range of about 0.001 to about 0.0001, about 0.001 to about 0.00001, about 0.001 to about 0.000001, about 0.0001 to about 0.00001, about 0.0001 to about 0.000001, or about 0.00001 to about 0.000001. In some embodiments, the threshold may be in the range of about 0.001, about 0.0001, about 0.00001, or about 0.000001. In some embodiments, the threshold may be in the range of at least about 0.001, about 0.0001, or about 0.00001. In some embodiments, the threshold may be in the range of at most about 0.0001, about 0.00001, or about 0.000001

### III. Corneal Topography Measurement Device

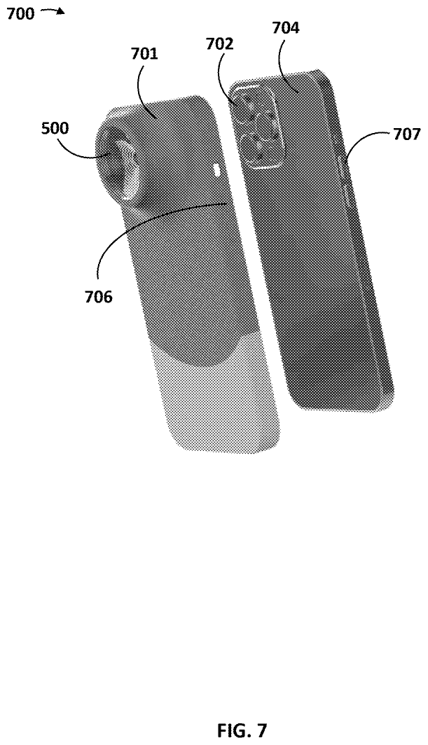

The systems, devices, and/or methods disclosed herein, in some embodiments, may comprise a device 700, as seen in FIG. 7, configured to measure corneal topography without the need of expensive keratometry machinery or specially trained clinical personnel. Due to the ubiquitous prevalence of mobile computing devices, the present invention can harness the combination of sensors and computational power of a mobile computing device platform. For example, many mobile computing devices (e.g., smart mobile phones) may comprise a camera, inertial sensors, light source (e.g., one or more light emitting diodes), onboard graphical processing unit and sufficient processing power to carry out image capture and processing with known spatial orientation.

In some embodiments, the device 700 may comprise an illumination body 500 optically and/or mechanically coupled to an imaging device 704. In some instances, the illumination body 500 may be housed, contained, and/or disposed in a structure 701, as shown in FIG. 7, configured to optically align and mechanically couple an imaging optical feature 504 of the illumination body 500 with a sensor 702 (e.g., a camera) of an imaging device 704 such that the sensor of the imaging device may collect, capture, and/or detect a reflected light pattern off of a subject's cornea. In some cases, the structure 701 may be configured to optically align and mechanically couple one or more input light coupling features 503 of the illumination 500 with one or more light source(s) of the imaging device. The structure 701 may be configured to be releasably coupled to the imaging device by a quick release mechanism that enables rapid attachment and/or removal of the structure and/or the illumination body from the imaging device. The quick release mechanism may comprise a snap-fit, lip and groove, hook and clasp, or any combination thereof mechanism.

In some cases, the structure 701, may comprise a rigid and/or a semi-rigid material configured to mechanically constrain one or more degrees of freedom of the illumination body with respect to the imaging device. In some cases, the structure 701 may comprise a mobile device (i.e., smartphone) case and/or skin configured to mate to the mobile device. In some instances, the structure 701 may comprise one or more regions 706 configured to transmit a user interaction (e.g., pressing a button and/or moving a slider) from the structure 701 to an input mechanical structure (e.g., button, slider, etc.) of the imaging device. In some instances, the input mechanical structure 707 of the imaging device may initiate, stop, pause, or any combination thereof actions, methods, and/or processes of the imaging device, described elsewhere herein. For example, the input mechanical structure 707 when engaged and/or interacted with by the user (e.g., pushing a button) may acquire one or more images and/or videos of a subject's cornea illuminated by the light pattern of the illumination body.

In some cases, the device 700 may be configured to measure and/or capture a reflected light pattern provided by illuminating the illumination body 500. The illumination body 500 may be illuminated by one or more light source(s) of the imaging device. In some cases, the one or more light sources of the imaging device may comprise one or more light emitting diodes (LEDs). The one or more light sources of the imaging device may comprise a flash of a mobile computing device (i.e., smartphone or smart cell phone). In some instances, the one or more light sources of the imaging device 704 may be optically coupled to the illumination body 500 by one or more input light coupling features 503 of the illumination body 500. The one or more input light coupling features 503 of the illumination body 500 may be configured to couple light emitted from the imaging device one or more light sources to the one or more features of the illumination body to generate a light pattern, described elsewhere herein. In some cases, the one or more light coupling features 503 of the illumination body 500 may optically couple light emitted from the one or more light coupling features normal (i.e., 90 degrees) to a surface of the imaging device to the one or more features of the illumination body to generate the light pattern. In some cases, the one or more light coupling features may comprise a reflective coating configured to reflect the emitted light of the imaging device one or more light sources to illuminate the features of the illumination body that generate the light pattern. In some embodiments, the one or more light coupling features 503 may comprise a prismatic cavity. In some embodiments, the one or more light coupling features 503 may comprise a waveguide that optically couples the emitted light of the imaging device's one or more light into the illumination body to illuminate the features of the illumination body to generate the light pattern. In some embodiments, the waveguide may comprise a lens-based system to couple the light from the imaging device's one or more light sources to the waveguide without losing light to total internal reflection between the interface of the one or more light sources and the waveguide. In some embodiments, up to 10%, up to 20%, up to 30%, up to 40% up to 50%, up to 60%, up to 70% up to 80%, up to 90% or up to 100% of the output light from the integrated illumination source may be coupled into the waveguide. In some embodiments, the lens-based system may comprise a ball lens, fish-eye lens, wide angle lens, macro lens, normal lens, or telephoto lens.

In some cases, the illumination body may comprise one or more features that may generate a light pattern when the illumination body is illuminated. In some instances, the light pattern generated by the illumination body 500 may comprise a depth sensing feature (502, 600, 602, 604, 606, 608, 610, 612, 614, 616) and a topography and/or curvature sensing feature 710. The depth sensing feature (502, 600, 602, 604, 606, 608, 610, 612, 614, 616) and a topography and/or curvature sensing feature 710 may be provided to a surface of a subject's cornea as a light pattern and collected and/or detected by a sensor (e.g., a camera) of the imaging device. The reflected light pattern collected off the subject's cornea may be compared with a known geometry and/or shape of the light pattern provided to measure corneal curvature at a given meridian.

In some embodiments, the topography and/or curvature sensing feature 710 may comprise one or more of lines and/or raised line features coupled to a surface of the illumination body 500. In some embodiments, the lines may comprise linear lines. In some embodiments, the lines may be circular, radial, parallel, converging, diverging, or any combination thereof. In some embodiments, the topography and/or curvature sensing feature 710 one or more lines and/or raised line features may comprise one or more rings and/or concentric ring structures. In some cases, the one or more lines and/or raised line features may comprise opaque line and/or raised line features.

In some embodiments, the lines may have a length of about 5 mm to about 30 mm. In some embodiments, the lines may have a length of about 5 mm to about 10 mm, about 5 mm to about 15 mm, about 5 mm to about 20 mm, about 5 mm to about 25 mm, about 5 mm to about 30 mm, about 10 mm to about 15 mm, about 10 mm to about 20 mm, about 10 mm to about 25 mm, about 10 mm to about 30 mm, about 15 mm to about 20 mm, about 15 mm to about 25 mm, about 15 mm to about 30 mm, about 20 mm to about 25 mm, about 20 mm to about 30 mm, or about 25 mm to about 30 mm. In some embodiments, the lines may have a length of about 5 mm, about 10 mm, about 15 mm, about 20 mm, about 25 mm, or about 30 mm. In some embodiments, the lines may have a length of at least about 5 mm, about 10 mm, about 15 mm, about 20 mm, or about 25 mm. In some embodiments, the lines may have a length of at most about 10 mm, about 15 mm, about 20 mm, about 25 mm, or about 30 mm.

In some embodiments, the lines may have a thickness of about 1 mm to about 10 mm. In some embodiments, the lines may have a thickness of about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 5 mm to about 6 mm, about 5 mm to about 7 mm, about 5 mm to about 8 mm, about 5 mm to about 9 mm, about 5 mm to about 10 mm, about 6 mm to about 7 mm, about 6 mm to about 8 mm, about 6 mm to about 9 mm, about 6 mm to about 10 mm, about 7 mm to about 8 mm, about 7 mm to about 9 mm, about 7 mm to about 10 mm, about 8 mm to about 9 mm, about 8 mm to about 10 mm, or about 9 mm to about 10 mm. In some embodiments, the lines may have a thickness of about 1 mm, about 2 mm, about 3 mm, about 4 mm, about 5 mm, about 6 mm, about 7 mm, about 8 mm, about 9 mm, or about 10 mm. In some embodiments, the lines may have a thickness of at least about 1 mm, about 2 mm, about 3 mm, about 4 mm, about 5 mm, about 6 mm, about 7 mm, about 8 mm, or about 9 mm.

In some embodiments, the lines may have an area of about 10 mm² to about 90 mm². In some embodiments, the lines may have an area of about 10 mm² to about 20 mm², about 10 mm² to about 30 mm², about 10 mm² to about 40 mm², about 10 mm² to about 50 mm², about 10 mm² to about 60 mm², about 10 mm² to about 70 mm², about 10 mm² to about 80 mm², about 10 mm² to about 90 mm², about 20 mm² to about 30 mm², about 20 mm² to about 40 mm², about 20 mm² to about 50 mm², about 20 mm² to about 60 mm², about 20 mm² to about 70 mm², about 20 mm² to about 80 mm², about 20 mm² to about 90 mm², about 30 mm² to about 40 mm², about 30 mm² to about 50 mm², about 30 mm² to about 60 mm², about 30 mm² to about 70 mm², about 30 mm² to about 80 mm², about 30 mm² to about 90 mm², about 40 mm² to about 50 mm², about 40 mm² to about 60 mm², about 40 mm² to about 70 mm², about 40 mm² to about 80 mm², about 40 mm² to about 90 mm², about 50 mm² to about 60 mm², about 50 mm² to about 70 mm², about 50 mm² to about 80 mm², about 50 mm² to about 90 mm², about 60 mm² to about 70 mm², about 60 mm² to about 80 mm², about 60 mm² to about 90 mm², about 70 mm² to about 80 mm², about 70 mm² to about 90 mm², or about 80 mm² to about 90 mm². In some embodiments, the lines may have an area of about 10 mm², about 20 mm², about 30 mm², about 40 mm², about 50 mm², about 60 mm², about 70 mm², about 80 mm², or about 90 mm². In some embodiments, the lines may have an area of at least about 10 mm², about 20 mm², about 30 mm², about 40 mm², about 50 mm², about 60 mm², about 70 mm², or about 80 mm². In some embodiments, the lines may have an area of at most about 20 mm², about 30 mm², about 40 mm², about 50 mm², about 60 mm², about 70 mm², about 80 mm², or about 90 mm².

In some embodiments, the lines may have a spacing from one another of about 1 mm to about 10 mm. In some embodiments, the lines may have a spacing from one another of about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 5 mm to about 6 mm, about 5 mm to about 7 mm, about 5 mm to about 8 mm, about 5 mm to about 9 mm, about 5 mm to about 10 mm, about 6 mm to about 7 mm, about 6 mm to about 8 mm, about 6 mm to about 9 mm, about 6 mm to about 10 mm, about 7 mm to about 8 mm, about 7 mm to about 9 mm, about 7 mm to about 10 mm, about 8 mm to about 9 mm, about 8 mm to about 10 mm, or about 9 mm to about 10 mm. In some embodiments, the lines may have a spacing from one another of about 1 mm, about 2 mm, about 3 mm, about 4 mm, about 5 mm, about 6 mm, about 7 mm, about 8 mm, about 9 mm, or about 10 mm. In some embodiments, the lines may have a spacing from one another of at least about 1 mm, about 2 mm, about 3 mm, about 4 mm, about 5 mm, about 6 mm, about 7 mm, about 8 mm, or about 9 mm. In some embodiments, the lines may have a spacing from one another of at most about 2 mm, about 3 mm, about 4 mm, about 5 mm, about 6 mm, about 7 mm, about 8 mm, about 9 mm, or about 10 mm.

In some embodiments, the illumination body 500 may be optically transparent or translucent. In some embodiments, the illumination body may comprise a material of polymethyl methacrylate (PMMA), acrylic, resin, clear resin, optical grade polycarbonate, or any combination thereof materials. In some embodiments, the illumination body may be opaque, transparent, or semi-transparent. The illumination body may be comprised of transparent or opaque segments. In some embodiments, the transparent or opaque segments may act as a wave guide to create an illuminated pattern.

In some embodiments, the illumination body 500 may comprise a curved and/or cone structure configured to fit inside an orbital socket of a subject's eye.

The illumination body may comprise a round and/or cylindrical structure with a diameter of about 28 mm to about 40 mm. The illumination body may comprise a round and/or cylindrical structure with a diameter of about 28 mm to about 30 mm, about 28 mm to about 31 mm, about 28 mm to about 32 mm, about 28 mm to about 33 mm, about 28 mm to about 34 mm, about 28 mm to about 35 mm, about 28 mm to about 36 mm, about 28 mm to about 37 mm, about 28 mm to about 38 mm, about 28 mm to about 39 mm, about 28 mm to about 40 mm, about 30 mm to about 31 mm, about 30 mm to about 32 mm, about 30 mm to about 33 mm, about 30 mm to about 34 mm, about 30 mm to about 35 mm, about 30 mm to about 36 mm, about 30 mm to about 37 mm, about 30 mm to about 38 mm, about 30 mm to about 39 mm, about 30 mm to about 40 mm, about 31 mm to about 32 mm, about 31 mm to about 33 mm, about 31 mm to about 34 mm, about 31 mm to about 35 mm, about 31 mm to about 36 mm, about 31 mm to about 37 mm, about 31 mm to about 38 mm, about 31 mm to about 39 mm, about 31 mm to about 40 mm, about 32 mm to about 33 mm, about 32 mm to about 34 mm, about 32 mm to about 35 mm, about 32 mm to about 36 mm, about 32 mm to about 37 mm, about 32 mm to about 38 mm, about 32 mm to about 39 mm, about 32 mm to about 40 mm, about 33 mm to about 34 mm, about 33 mm to about 35 mm, about 33 mm to about 36 mm, about 33 mm to about 37 mm, about 33 mm to about 38 mm, about 33 mm to about 39 mm, about 33 mm to about 40 mm, about 34 mm to about 35 mm, about 34 mm to about 36 mm, about 34 mm to about 37 mm, about 34 mm to about 38 mm, about 34 mm to about 39 mm, about 34 mm to about 40 mm, about 35 mm to about 36 mm, about 35 mm to about 37 mm, about 35 mm to about 38 mm, about 35 mm to about 39 mm, about 35 mm to about 40 mm, about 36 mm to about 37 mm, about 36 mm to about 38 mm, about 36 mm to about 39 mm, about 36 mm to about 40 mm, about 37 mm to about 38 mm, about 37 mm to about 39 mm, about 37 mm to about 40 mm, about 38 mm to about 39 mm, about 38 mm to about 40 mm, or about 39 mm to about 40 mm. The illumination body may comprise a round and/or cylindrical structure with a diameter of about 28 mm, about 30 mm, about 31 mm, about 32 mm, about 33 mm, about 34 mm, about 35 mm, about 36 mm, about 37 mm, about 38 mm, about 39 mm, or about 40 mm. The illumination body may comprise a round and/or cylindrical structure with a diameter of at least about 28 mm, about 30 mm, about 31 mm, about 32 mm, about 33 mm, about 34 mm, about 35 mm, about 36 mm, about 37 mm, about 38 mm, or about 39 mm. The illumination body may comprise a round and/or cylindrical structure with a diameter of at most about 30 mm, about 31 mm, about 32 mm, about 33 mm, about 34 mm, about 35 mm, about 36 mm, about 37 mm, about 38 mm, about 39 mm, or about 40 mm.

In some embodiments, the illumination body may comprise segments of material with high scattering coefficient. In some embodiments, the segments of the illumination body of high scattering coefficient may create a light pattern. In some embodiments, the illumination body may have a refractive index of about 1.3 to about 1.5. In some embodiments, the illumination body may have a refractive index of about 1.3 to about 1.32, about 1.3 to about 1.34, about 1.3 to about 1.36, about 1.3 to about 1.38, about 1.3 to about 1.4, about 1.3 to about 1.42, about 1.3 to about 1.44, about 1.3 to about 1.46, about 1.3 to about 1.48, about 1.3 to about 1.5, about 1.32 to about 1.34, about 1.32 to about 1.36, about 1.32 to about 1.38, about 1.32 to about 1.4, about 1.32 to about 1.42, about 1.32 to about 1.44, about 1.32 to about 1.46, about 1.32 to about 1.48, about 1.32 to about 1.5, about 1.34 to about 1.36, about 1.34 to about 1.38, about 1.34 to about 1.4, about 1.34 to about 1.42, about 1.34 to about 1.44, about 1.34 to about 1.46, about 1.34 to about 1.48, about 1.34 to about 1.5, about 1.36 to about 1.38, about 1.36 to about 1.4, about 1.36 to about 1.42, about 1.36 to about 1.44, about 1.36 to about 1.46, about 1.36 to about 1.48, about 1.36 to about 1.5, about 1.38 to about 1.4, about 1.38 to about 1.42, about 1.38 to about 1.44, about 1.38 to about 1.46, about 1.38 to about 1.48, about 1.38 to about 1.5, about 1.4 to about 1.42, about 1.4 to about 1.44, about 1.4 to about 1.46, about 1.4 to about 1.48, about 1.4 to about 1.5, about 1.42 to about 1.44, about 1.42 to about 1.46, about 1.42 to about 1.48, about 1.42 to about 1.5, about 1.44 to about 1.46, about 1.44 to about 1.48, about 1.44 to about 1.5, about 1.46 to about 1.48, about 1.46 to about 1.5, or about 1.48 to about 1.5. In some embodiments, the illumination body may have a refractive index of about 1.3, about 1.32, about 1.34, about 1.36, about 1.38, about 1.4, about 1.42, about 1.44, about 1.46, about 1.48, or about 1.5. In some embodiments, the illumination body may have a refractive index of at least about 1.3, about 1.32, about 1.34, about 1.36, about 1.38, about 1.4, about 1.42, about 1.44, about 1.46, or about 1.48. In some embodiments, the illumination body may have a refractive index of at most about 1.32, about 1.34, about 1.36, about 1.38, about 1.4, about 1.42, about 1.44, about 1.46, about 1.48, or about 1.5.

In some embodiments, the illumination body may have an absorption coefficient of about 1 cm\hat{ }−1 to about 10 cm\hat{ }−1. In some embodiments, the illumination body may have an absorption coefficient of about 1 cm\hat{ }−1 to about 2 cm\hat{ }−1, about 1 cm\hat{ }−1 to about 3 cm\hat{ }−1, about 1 cm\hat{ }−1 to about 4 cm\hat{ }−1, about 1 cm\hat{ }−1 to about 5 cm\hat{ }−1, about 1 cm\hat{ }−1 to about 6 cm\hat{ }−1, about 1 cm\hat{ }−1 to about 7 cm\hat{ }−1, about 1 cm\hat{ }−1 to about 8 cm\hat{ }−1, about 1 cm\hat{ }−1 to about 9 cm\hat{ }−1, about 1 cm\hat{ }−1 to about 10 cm\hat{ }−1, about 2 cm\hat{ }−1 to about 3 cm\hat{ }−1, about 2 cm\hat{ }−1 to about 4 cm\hat{ }−1, about 2 cm\hat{ }−1 to about 5 cm\hat{ }−1, about 2 cm\hat{ }−1 to about 6 cm\hat{ }−1, about 2 cm\hat{ }−1 to about 7 cm\hat{ }−1, about 2 cm\hat{ }−1 to about 8 cm\hat{ }−1, about 2 cm\hat{ }−1 to about 9 cm\hat{ }−1, about 2 cm\hat{ }−1 to about 10 cm\hat{ }−1, about 3 cm\hat{ }−1 to about 4 cm\hat{ }−1, about 3 cm\hat{ }−1 to about 5 cm\hat{ }−1, about 3 cm\hat{ }−1 to about 6 cm\hat{ }−1, about 3 cm\hat{ }−1 to about 7 cm\hat{ }−1, about 3 cm\hat{ }−1 to about 8 cm\hat{ }−1, about 3 cm\hat{ }−1 to about 9 cm\hat{ }−1, about 3 cm\hat{ }−1 to about 10 cm\hat{ }−1, about 4 cm\hat{ }−1 to about 5 cm\hat{ }−1, about 4 cm\hat{ }−1 to about 6 cm\hat{ }−1, about 4 cm\hat{ }−1 to about 7 cm\hat{ }−1, about 4 cm\hat{ }−1 to about 8 cm\hat{ }−1, about 4 cm\hat{ }−1 to about 9 cm\hat{ }−1, about 4 cm\hat{ }−1 to about 10 cm\hat{ }−1, about 5 cm\hat{ }−1 to about 6 cm\hat{ }−1, about 5 cm\hat{ }−1 to about 7 cm\hat{ }−1, about 5 cm\hat{ }−1 to about 8 cm\hat{ }−1, about 5 cm\hat{ }−1 to about 9 cm\hat{ }−1, about 5 cm\hat{ }−1 to about 10 cm\hat{ }−1, about 6 cm\hat{ }−1 to about 7 cm\hat{ }−1, about 6 cm\hat{ }−1 to about 8 cm\hat{ }−1, about 6 cm\hat{ }−1 to about 9 cm\hat{ }−1, about 6 cm\hat{ }−1 to about 10 cm\hat{ }−1, about 7 cm\hat{ }−1 to about 8 cm\hat{ }−1, about 7 cm\hat{ }−1 to about 9 cm\hat{ }−1, about 7 cm\hat{ }−1 to about 10 cm\hat{ }−1, about 8 cm\hat{ }−1 to about 9 cm\hat{ }−1, about 8 cm\hat{ }−1 to about 10 cm\hat{ }−1, or about 9 cm\hat{ }−1 to about 10 cm\hat{ }−1. In some embodiments, the illumination body may have an absorption coefficient of about 1 cm\hat{ }−1, about 2 cm\hat{ }−1, about 3 cm\hat{ }−1, about 4 cm\hat{ }−1, about 5 cm\hat{ }−1, about 6 cm\hat{ }−1, about 7 cm\hat{ }−1, about 8 cm\hat{ }−1, about 9 cm\hat{ }−1, or about 10 cm\hat{ }−1. In some embodiments, the illumination body may have an absorption coefficient of at least about 1 cm\hat{ }−1, about 2 cm\hat{ }−1, about 3 cm\hat{ }−1, about 4 cm\hat{ }−1, about 5 cm\hat{ }−1, about 6 cm\hat{ }−1, about 7 cm\hat{ }−1, about 8 cm\hat{ }−1, or about 9 cm\hat{ }−1. In some embodiments, the illumination body may have an absorption coefficient of at most about 2 cm\hat{ }−1, about 3 cm\hat{ }−1, about 4 cm\hat{ }−1, about 5 cm\hat{ }−1, about 6 cm\hat{ }−1, about 7 cm\hat{ }−1, about 8 cm\hat{ }−1, about 9 cm\hat{ }−1, or about 10 cm\hat{ }−1.

In some cases, an outer surface of the illumination body 500 may comprise a coating configured to reflect the coupled light of the one or more light sources of the imaging device internally within the illumination body to generate a diffuse uniform illumination light pattern, described elsewhere herein. In some instances, the coating may comprise a metal coating. The metal coating may comprise a gold, silver, aluminum, titanium, or any combination thereof coating.

In some cases, the illumination body 500 may provide uniform illumination of light pattern to a target (e.g., a surface of a subject's cornea) to improve the determination of shape, spatial topography, and/or contour of a subject's cornea. In some cases, uniform illumination comprises an illumination of one or more regions of a target (e.g., a surface of a subject's cornea) such that a first region of the target has a brightness that is within about 10%, 9%, 8%, 6%, 4%, 2%, 1%, or less of a brightness of a second region of the target. In some cases, the brightness in a first region may be the same or similar to the brightness in a second region. In some instances, the target may comprise a biological tissue e.g., a corneal surface. In some instances, the corneal surface may be a mammalian corneal surface. In some cases, the illumination body may be configured to produce a uniform light pattern from a point light source. In some instances, the uniform light pattern may comprise a light source configured to provide an emitted illumination such that the emitted light source may comprise a constant or near constant brightness and/or intensity across an area, surface, field-of-view, or any combination thereof. In some cases, a point light source may comprise one or more light sources of the imaging device, described elsewhere herein.

In some cases, the one or more light coupling features of the illumination body may be configured to receive and distribute light from the one or more light sources of the imaging device to the plurality of light scattering elements of the illumination body while reducing or minimizing a light hot spot at or near emitted light of the one or more light sources of the imaging device. In some instances, the light hot spot may correspond to or refer to a concentration of light at or near the one or more light sources of the imaging device. In some cases, the refractive index of one or more light coupling features may comprise a different refractive index than that of the illumination body. In some instances, the one or more light coupling features may direct the light emitted by the light source towards the one or more light scattering elements of the illumination body. In some cases, a geometry of the one or more light coupling features may be determined by the geometry and light emission characters of the one or more light sources of the imaging device. For example, two light sources with varying etendue may require a variation of the geometry of the one or more light coupling features.

In some cases, the illumination body 500 described herein may comprise one or more light scattering elements disposed a surface of the illumination body. In some instances, the one or more light scattering elements may comprise a two-dimensional array of light scattering elements. In some instances, the two-dimensional array of the one or more light scattering elements may comprise a linear configuration, non-linear configuration, or any combination thereof. In some cases, the one or more light scattering elements may comprise one or more dome reflectors, scattering particles, prisms, prisms, mirrors, dome reflectors, or any combination thereof.

In some cases, the curvature or composition of the light scattering elements may provide a geometric or light scattering re-distribution and/or re-direction of incident light onto the one or more scattering elements. In some instances, the one or more light scattering elements may isotopically scatter or re-distribute incident photons. In some instances, the light scattering elements may participate in Raleigh, Mie, or any combination thereof light scattering interactions. In some instances, the light scattering interaction with incident light may be determined by the difference in refractive index between the light scattering element and the surrounding medium.

The refractive index different between the light scattering element and the surround medium of the illumination body may comprise a refractive index difference of about 0.1 to about 2. The refractive index different between the light scattering element and the surround medium of the illumination body may comprise a refractive index difference of about 0.1 to about 0.2, about 0.1 to about 0.3, about 0.1 to about 0.4, about 0.1 to about 0.5, about 0.1 to about 0.6, about 0.1 to about 0.7, about 0.1 to about 0.8, about 0.1 to about 0.9, about 0.1 to about 1, about 0.1 to about 1.5, about 0.1 to about 2, about 0.2 to about 0.3, about 0.2 to about 0.4, about 0.2 to about 0.5, about 0.2 to about 0.6, about 0.2 to about 0.7, about 0.2 to about 0.8, about 0.2 to about 0.9, about 0.2 to about 1, about 0.2 to about 1.5, about 0.2 to about 2, about 0.3 to about 0.4, about 0.3 to about 0.5, about 0.3 to about 0.6, about 0.3 to about 0.7, about 0.3 to about 0.8, about 0.3 to about 0.9, about 0.3 to about 1, about 0.3 to about 1.5, about 0.3 to about 2, about 0.4 to about 0.5, about 0.4 to about 0.6, about 0.4 to about 0.7, about 0.4 to about 0.8, about 0.4 to about 0.9, about 0.4 to about 1, about 0.4 to about 1.5, about 0.4 to about 2, about 0.5 to about 0.6, about 0.5 to about 0.7, about 0.5 to about 0.8, about 0.5 to about 0.9, about 0.5 to about 1, about 0.5 to about 1.5, about 0.5 to about 2, about 0.6 to about 0.7, about 0.6 to about 0.8, about 0.6 to about 0.9, about 0.6 to about 1, about 0.6 to about 1.5, about 0.6 to about 2, about 0.7 to about 0.8, about 0.7 to about 0.9, about 0.7 to about 1, about 0.7 to about 1.5, about 0.7 to about 2, about 0.8 to about 0.9, about 0.8 to about 1, about 0.8 to about 1.5, about 0.8 to about 2, about 0.9 to about 1, about 0.9 to about 1.5, about 0.9 to about 2, about 1 to about 1.5, about 1 to about 2, or about 1.5 to about 2. The refractive index different between the light scattering element and the surround medium of the illumination body may comprise a refractive index difference of about 0.1, about 0.2, about 0.3, about 0.4, about 0.5, about 0.6, about 0.7, about 0.8, about 0.9, about 1, about 1.5, or about 2. The refractive index different between the light scattering element and the surround medium of the illumination body may comprise a refractive index difference of at least about 0.1, about 0.2, about 0.3, about 0.4, about 0.5, about 0.6, about 0.7, about 0.8, about 0.9, about 1, or about 1.5. The refractive index different between the light scattering element and the surround medium of the illumination body may comprise a refractive index difference of at most about 0.2, about 0.3, about 0.4, about 0.5, about 0.6, about 0.7, about 0.8, about 0.9, about 1, about 1.5, or about 2.

In some cases, the light scattering elements may comprise a geometry configured to re-direct or re-distribute incident photons in all or a subset thereof spherical vector trajectories of reflected or scattered light between the incident light and the one or more light scattering elements. The one or more light scattering elements may diffuse and/or re-distribute light in a way to generate uniform light pattern emitted from the illumination body.

In some instances, the domed reflectors may comprise a radius of about 0.1 mm to about 1.5 mm. In some instances, the domed reflectors 108 may comprise a radius of about 0.1 mm to about 0.2 mm, about 0.1 mm to about 0.3 mm, about 0.1 mm to about 0.4 mm, about 0.1 mm to about 0.5 mm, about 0.1 mm to about 0.6 mm, about 0.1 mm to about 0.7 mm, about 0.1 mm to about 0.8 mm, about 0.1 mm to about 0.9 mm, about 0.1 mm to about 1 mm, about 0.1 mm to about 1.2 mm, about 0.1 mm to about 1.5 mm, about 0.2 mm to about 0.3 mm, about 0.2 mm to about 0.4 mm, about 0.2 mm to about 0.5 mm, about 0.2 mm to about 0.6 mm, about 0.2 mm to about 0.7 mm, about 0.2 mm to about 0.8 mm, about 0.2 mm to about 0.9 mm, about 0.2 mm to about 1 mm, about 0.2 mm to about 1.2 mm, about 0.2 mm to about 1.5 mm, about 0.3 mm to about 0.4 mm, about 0.3 mm to about 0.5 mm, about 0.3 mm to about 0.6 mm, about 0.3 mm to about 0.7 mm, about 0.3 mm to about 0.8 mm, about 0.3 mm to about 0.9 mm, about 0.3 mm to about 1 mm, about 0.3 mm to about 1.2 mm, about 0.3 mm to about 1.5 mm, about 0.4 mm to about 0.5 mm, about 0.4 mm to about 0.6 mm, about 0.4 mm to about 0.7 mm, about 0.4 mm to about 0.8 mm, about 0.4 mm to about 0.9 mm, about 0.4 mm to about 1 mm, about 0.4 mm to about 1.2 mm, about 0.4 mm to about 1.5 mm, about 0.5 mm to about 0.6 mm, about 0.5 mm to about 0.7 mm, about 0.5 mm to about 0.8 mm, about 0.5 mm to about 0.9 mm, about 0.5 mm to about 1 mm, about 0.5 mm to about 1.2 mm, about 0.5 mm to about 1.5 mm, about 0.6 mm to about 0.7 mm, about 0.6 mm to about 0.8 mm, about 0.6 mm to about 0.9 mm, about 0.6 mm to about 1 mm, about 0.6 mm to about 1.2 mm, about 0.6 mm to about 1.5 mm, about 0.7 mm to about 0.8 mm, about 0.7 mm to about 0.9 mm, about 0.7 mm to about 1 mm, about 0.7 mm to about 1.2 mm, about 0.7 mm to about 1.5 mm, about 0.8 mm to about 0.9 mm, about 0.8 mm to about 1 mm, about 0.8 mm to about 1.2 mm, about 0.8 mm to about 1.5 mm, about 0.9 mm to about 1 mm, about 0.9 mm to about 1.2 mm, about 0.9 mm to about 1.5 mm, about 1 mm to about 1.2 mm, about 1 mm to about 1.5 mm, or about 1.2 mm to about 1.5 mm. In some instances, the domed reflectors 1108 may comprise a radius of about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.2 mm, or about 1.5 mm. In some instances, the domed reflectors may comprise a radius of at least about 0.1 mm, about 0.2 mm, about 0.3 mm, about 0.4 mm, about 0.5 mm, about 0.6 mm, about 0.7 mm, about 0.8 mm, about 0.9 mm, about 1 mm, or about 1.2 mm. In some instances, the domed reflectors 108 may comprise a radius of at most about 0.2 mm, about 0.3 mm, about 0.4 mm, about 0.5 mm, about 0.6 mm, about 0.7 mm, about 0.8 mm, about 0.9 mm, about 1 mm, about 1.2 mm, or about 1.5 mm.

In some cases, the domed reflectors may comprise a depth of about 0.1 mm to about 1.5 mm. In some cases, the domed reflectors 108 may comprise a depth of about 0.1 mm to about 0.2 mm, about 0.1 mm to about 0.3 mm, about 0.1 mm to about 0.4 mm, about 0.1 mm to about 0.5 mm, about 0.1 mm to about 0.6 mm, about 0.1 mm to about 0.7 mm, about 0.1 mm to about 0.8 mm, about 0.1 mm to about 0.9 mm, about 0.1 mm to about 1 mm, about 0.1 mm to about 1.2 mm, about 0.1 mm to about 1.5 mm, about 0.2 mm to about 0.3 mm, about 0.2 mm to about 0.4 mm, about 0.2 mm to about 0.5 mm, about 0.2 mm to about 0.6 mm, about 0.2 mm to about 0.7 mm, about 0.2 mm to about 0.8 mm, about 0.2 mm to about 0.9 mm, about 0.2 mm to about 1 mm, about 0.2 mm to about 1.2 mm, about 0.2 mm to about 1.5 mm, about 0.3 mm to about 0.4 mm, about 0.3 mm to about 0.5 mm, about 0.3 mm to about 0.6 mm, about 0.3 mm to about 0.7 mm, about 0.3 mm to about 0.8 mm, about 0.3 mm to about 0.9 mm, about 0.3 mm to about 1 mm, about 0.3 mm to about 1.2 mm, about 0.3 mm to about 1.5 mm, about 0.4 mm to about 0.5 mm, about 0.4 mm to about 0.6 mm, about 0.4 mm to about 0.7 mm, about 0.4 mm to about 0.8 mm, about 0.4 mm to about 0.9 mm, about 0.4 mm to about 1 mm, about 0.4 mm to about 1.2 mm, about 0.4 mm to about 1.5 mm, about 0.5 mm to about 0.6 mm, about 0.5 mm to about 0.7 mm, about 0.5 mm to about 0.8 mm, about 0.5 mm to about 0.9 mm, about 0.5 mm to about 1 mm, about 0.5 mm to about 1.2 mm, about 0.5 mm to about 1.5 mm, about 0.6 mm to about 0.7 mm, about 0.6 mm to about 0.8 mm, about 0.6 mm to about 0.9 mm, about 0.6 mm to about 1 mm, about 0.6 mm to about 1.2 mm, about 0.6 mm to about 1.5 mm, about 0.7 mm to about 0.8 mm, about 0.7 mm to about 0.9 mm, about 0.7 mm to about 1 mm, about 0.7 mm to about 1.2 mm, about 0.7 mm to about 1.5 mm, about 0.8 mm to about 0.9 mm, about 0.8 mm to about 1 mm, about 0.8 mm to about 1.2 mm, about 0.8 mm to about 1.5 mm, about 0.9 mm to about 1 mm, about 0.9 mm to about 1.2 mm, about 0.9 mm to about 1.5 mm, about 1 mm to about 1.2 mm, about 1 mm to about 1.5 mm, or about 1.2 mm to about 1.5 mm. In some cases, the domed reflectors 108 may comprise a depth of about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.2 mm, or about 1.5 mm. In some cases, the domed reflectors may comprise a depth of at least about 0.1 mm, about 0.2 mm, about 0.3 mm, about 0.4 mm, about 0.5 mm, about 0.6 mm, about 0.7 mm, about 0.8 mm, about 0.9 mm, about 1 mm, or about 1.2 mm. In some cases, the domed reflectors may comprise a depth of at most about 0.2 mm, about 0.3 mm, about 0.4 mm, about 0.5 mm, about 0.6 mm, about 0.7 mm, about 0.8 mm, about 0.9 mm, about 1 mm, about 1.2 mm, or about 1.5 mm.

In some embodiments, the illumination body 500 may comprise a depth sensing feature (502, 600, 602, 604, 606, 608, 610, 612, 614, 616), as seen in FIGS. 5A-5B and FIGS. 6A-6E. In some instances, the depth sensing feature may comprise an angled surface 612 with respect a planar surface 613 of the illumination body 500, as seen in FIG. 6A. The angled surface 612 may provide a region of the illumination where light coupled from the imaging device one or more light sources may reflect and diffuse to a uniform illumination prior to exiting the illumination body as the light pattern. In some cases, the angled surface 612 may comprise an angle of about 28 degrees to about 50 degrees with respect to the planar surface 613 of the illumination body 500. In some cases, the angled surface 612 may comprise an angle of about 28 degrees to about 30 degrees, about 28 degrees to about 32 degrees, about 28 degrees to about 34 degrees, about 28 degrees to about 36 degrees, about 28 degrees to about 38 degrees, about 28 degrees to about 40 degrees, about 28 degrees to about 42 degrees, about 28 degrees to about 44 degrees, about 28 degrees to about 46 degrees, about 28 degrees to about 48 degrees, about 28 degrees to about 50 degrees, about 30 degrees to about 32 degrees, about 30 degrees to about 34 degrees, about 30 degrees to about 36 degrees, about 30 degrees to about 38 degrees, about 30 degrees to about 40 degrees, about 30 degrees to about 42 degrees, about 30 degrees to about 44 degrees, about 30 degrees to about 46 degrees, about 30 degrees to about 48 degrees, about 30 degrees to about 50 degrees, about 32 degrees to about 34 degrees, about 32 degrees to about 36 degrees, about 32 degrees to about 38 degrees, about 32 degrees to about 40 degrees, about 32 degrees to about 42 degrees, about 32 degrees to about 44 degrees, about 32 degrees to about 46 degrees, about 32 degrees to about 48 degrees, about 32 degrees to about 50 degrees, about 34 degrees to about 36 degrees, about 34 degrees to about 38 degrees, about 34 degrees to about 40 degrees, about 34 degrees to about 42 degrees, about 34 degrees to about 44 degrees, about 34 degrees to about 46 degrees, about 34 degrees to about 48 degrees, about 34 degrees to about 50 degrees, about 36 degrees to about 38 degrees, about 36 degrees to about 40 degrees, about 36 degrees to about 42 degrees, about 36 degrees to about 44 degrees, about 36 degrees to about 46 degrees, about 36 degrees to about 48 degrees, about 36 degrees to about 50 degrees, about 38 degrees to about 40 degrees, about 38 degrees to about 42 degrees, about 38 degrees to about 44 degrees, about 38 degrees to about 46 degrees, about 38 degrees to about 48 degrees, about 38 degrees to about 50 degrees, about 40 degrees to about 42 degrees, about 40 degrees to about 44 degrees, about 40 degrees to about 46 degrees, about 40 degrees to about 48 degrees, about 40 degrees to about 50 degrees, about 42 degrees to about 44 degrees, about 42 degrees to about 46 degrees, about 42 degrees to about 48 degrees, about 42 degrees to about 50 degrees, about 44 degrees to about 46 degrees, about 44 degrees to about 48 degrees, about 44 degrees to about 50 degrees, about 46 degrees to about 48 degrees, about 46 degrees to about 50 degrees, or about 48 degrees to about 50 degrees. In some cases, the angled surface 612 may comprise an angle of about 28 degrees, about 30 degrees, about 32 degrees, about 34 degrees, about 36 degrees, about 38 degrees, about 40 degrees, about 42 degrees, about 44 degrees, about 46 degrees, about 48 degrees, or about 50 degrees. In some cases, the angled surface 612 may comprise an angle of at least about 28 degrees, about 30 degrees, about 32 degrees, about 34 degrees, about 36 degrees, about 38 degrees, about 40 degrees, about 42 degrees, about 44 degrees, about 46 degrees, or about 48 degrees. In some cases, the angled surface 612 may comprise an angle of at most about 30 degrees, about 32 degrees, about 34 degrees, about 36 degrees, about 38 degrees, about 40 degrees, about 42 degrees, about 44 degrees, about 46 degrees, about 48 degrees, or about 50 degrees.

In some cases, the depth sensing feature (502, 600, 602, 604, 606, 608, 610, 612, 614, 616) may comprise a first region that may permit and/or transmit light (600, 604, 608, 612) and/or a second region that may block, attenuate, and/or filter (602, 610, 606, 614, 616) one or more wavelengths of the emitted light of the one or more light sources of the imaging device prior to exiting the illumination body as a light pattern. In some instances, the depth sensing feature (608, 610), as shown in FIG. 6D, may comprise an array of one or more alternating first regions 608 and/or second regions 610 of depth sensing features. In some cases, the depth sensing features (614, 612) may be configured to filter the emitted light of the one or more light sources of the imaging device to shift the output wavelength of the depth sensing feature of the light pattern, as shown in FIG. 6E. In some cases, the filter may be configured to filter the emitting light of the one or more light sources to a visible color wavelength e.g., red, green, blue, violate, orange, and/or yellow prior to emitting the filtered light as a light pattern from the illumination body. The resulting light pattern emitted from the illumination body of the depth sensing feature may comprise a combination of the first region and the second region, where the contrast and/or difference in light signal between the first region and/or the second region of the depth sensing feature overlayed on the topography and/or curvature sensing feature 710 may provide a distance of the illumination body and/or the imaging device from a subject's cornea.

In some instances, the depth sensing feature may comprise one or more shapes and/or geometries. The one or more shapes and/or geometries may comprise linear (600, 602, 608, 610, 612, 614), circular (606, 604), and/or any other polygonal shape. In some cases, the circular depth sensing feature shapes may comprise concentric and/or co-linear circular first and second region shapes, as seen in FIG. 6C. In some cases, the depth sensing feature may comprise one or more shapes and/or geometries arranged at a fixed angle e.g., two depth sensing feature shapes placed 180 degrees apart (600, 602, 608, 610), as shown in FIGS. 6A and 6D, and/or four depth sensing feature shapes placed 90 degrees apart, as shown in FIG. 6B. In some cases, the depth sensing feature may comprise three depth sensing feature shapes placed 120 degrees apart.

In some embodiments, the depth sensing feature (502, 600, 602, 604, 606, 608, 610, 612, 614, 616) may comprise a width and a depth. The depth may be measured from a top or a bottom surface of the illumination body to a corresponding highest and/or lowest point of the angled depth sensing feature. In some instances, the depth of the depth sensing feature may alter the sensitivity of the measurement of the distance between the illumination body and/or imaging device and the subject's cornea. For example, the deeper depth sensing feature may provide a greater sensitivity in the determination of the distance between the illumination body and/or imaging device from the cornea compared to a shallow depth sensing feature. In some cases, the width of the depth sensing feature may alter the accuracy of determining and/or detecting the position of the depth sensing feature overlaid on the light pattern generated by the topography and/or curvature sensing feature 710. For example, a thin depth sensing feature may provide more information and/or data to the rotation of the illumination body and/or imaging device towards and/or away from a plane normal to the surface of the subject's cornea. A thicker depth sensing feature on the other hand may provide a greater area for detection by one or more processes and/or algorithms, described elsewhere herein.

In some instances, the width of the depth sensing feature may be about 1 mm to about 12 mm. In some instances, the width of the depth sensing feature may be about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 1 mm to about 11 mm, about 1 mm to about 12 mm, about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 2 mm to about 11 mm, about 2 mm to about 12 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 3 mm to about 11 mm, about 3 mm to about 12 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 4 mm to about 11 mm, about 4 mm to about 12 mm, about 5 mm to about 6 mm, about 5 mm to about 7 mm, about 5 mm to about 8 mm, about 5 mm to about 9 mm, about 5 mm to about 10 mm, about 5 mm to about 11 mm, about 5 mm to about 12 mm, about 6 mm to about 7 mm, about 6 mm to about 8 mm, about 6 mm to about 9 mm, about 6 mm to about 10 mm, about 6 mm to about 11 mm, about 6 mm to about 12 mm, about 7 mm to about 8 mm, about 7 mm to about 9 mm, about 7 mm to about 10 mm, about 7 mm to about 11 mm, about 7 mm to about 12 mm, about 8 mm to about 9 mm, about 8 mm to about 10 mm, about 8 mm to about 11 mm, about 8 mm to about 12 mm, about 9 mm to about 10 mm, about 9 mm to about 11 mm, about 9 mm to about 12 mm, about 10 mm to about 11 mm, about 10 mm to about 12 mm, or about 11 mm to about 12 mm. In some instances, the width of the depth sensing feature may be about 1 mm, about 2 mm, about 3 mm, about 4 mm, about 5 mm, about 6 mm, about 7 mm, about 8 mm, about 9 mm, about 10 mm, about 11 mm, or about 12 mm. In some instances, the width of the depth sensing feature may be at least about 1 mm, about 2 mm, about 3 mm, about 4 mm, about 5 mm, about 6 mm, about 7 mm, about 8 mm, about 9 mm, about 10 mm, or about 11 mm. In some instances, the width of the depth sensing feature may be at most about 2 mm, about 3 mm, about 4 mm, about 5 mm, about 6 mm, about 7 mm, about 8 mm, about 9 mm, about 10 mm, about 11 mm, or about 12 mm.

In some cases, the depth of the depth sensing feature may be about 1 mm to about 12 mm. In some cases, the depth of the depth sensing feature may be about 1 mm to about 2 mm, about 1 mm to about 3 mm, about 1 mm to about 4 mm, about 1 mm to about 5 mm, about 1 mm to about 6 mm, about 1 mm to about 7 mm, about 1 mm to about 8 mm, about 1 mm to about 9 mm, about 1 mm to about 10 mm, about 1 mm to about 11 mm, about 1 mm to about 12 mm, about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 2 mm to about 11 mm, about 2 mm to about 12 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 3 mm to about 11 mm, about 3 mm to about 12 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 4 mm to about 11 mm, about 4 mm to about 12 mm, about 5 mm to about 6 mm, about 5 mm to about 7 mm, about 5 mm to about 8 mm, about 5 mm to about 9 mm, about 5 mm to about 10 mm, about 5 mm to about 11 mm, about 5 mm to about 12 mm, about 6 mm to about 7 mm, about 6 mm to about 8 mm, about 6 mm to about 9 mm, about 6 mm to about 10 mm, about 6 mm to about 11 mm, about 6 mm to about 12 mm, about 7 mm to about 8 mm, about 7 mm to about 9 mm, about 7 mm to about 10 mm, about 7 mm to about 11 mm, about 7 mm to about 12 mm, about 8 mm to about 9 mm, about 8 mm to about 10 mm, about 8 mm to about 11 mm, about 8 mm to about 12 mm, about 9 mm to about 10 mm, about 9 mm to about 11 mm, about 9 mm to about 12 mm, about 10 mm to about 11 mm, about 10 mm to about 12 mm, or about 11 mm to about 12 mm. In some cases, the depth of the depth sensing feature may be about 1 mm, about 2 mm, about 3 mm, about 4 mm, about 5 mm, about 6 mm, about 7 mm, about 8 mm, about 9 mm, about 10 mm, about 11 mm, or about 12 mm. In some cases, the depth of the depth sensing feature may be at least about 1 mm, about 2 mm, about 3 mm, about 4 mm, about 5 mm, about 6 mm, about 7 mm, about 8 mm, about 9 mm, about 10 mm, or about 11 mm. In some cases, the depth of the depth sensing feature may be at most about 2 mm, about 3 mm, about 4 mm, about 5 mm, about 6 mm, about 7 mm, about 8 mm, about 9 mm, about 10 mm, about 11 mm, or about 12 mm.

In some embodiments, the imaging device may comprise a sensor (e.g., camera) that provides an optical power of about 5 diopters to about 40 diopters. In some embodiments, the optical power may be about 5 diopters to about 10 diopters, about 5 diopters to about 15 diopters, about 5 diopters to about 20 diopters, about 5 diopters to about 25 diopters, about 5 diopters to about 30 diopters, about 5 diopters to about 35 diopters, about 5 diopters to about 40 diopters, about 10 diopters to about 15 diopters, about 10 diopters to about 20 diopters, about 10 diopters to about 25 diopters, about 10 diopters to about 30 diopters, about 10 diopters to about 35 diopters, about 10 diopters to about 40 diopters, about 15 diopters to about 20 diopters, about 15 diopters to about 25 diopters, about 15 diopters to about 30 diopters, about 15 diopters to about 35 diopters, about 15 diopters to about 40 diopters, about 20 diopters to about 25 diopters, about 20 diopters to about 30 diopters, about 20 diopters to about 35 diopters, about 20 diopters to about 40 diopters, about 25 diopters to about 30 diopters, about 25 diopters to about 35 diopters, about 25 diopters to about 40 diopters, about 30 diopters to about 35 diopters, about 30 diopters to about 40 diopters, or about 35 diopters to about 40 diopters. In some embodiments, the optical power may be about 5 diopters, about 10 diopters, about 15 diopters, about 20 diopters, about 25 diopters, about 30 diopters, about 35 diopters, or about 40 diopters. In some embodiments, the optical power may be at least about 5 diopters, about 10 diopters, about 15 diopters, about 20 diopters, about 25 diopters, about 30 diopters, or about 35 diopters. In some embodiments, the optical power may be at most about 10 diopters, about 15 diopters, about 20 diopters, about 25 diopters, about 30 diopters, about 35 diopters, or about 40 diopters.

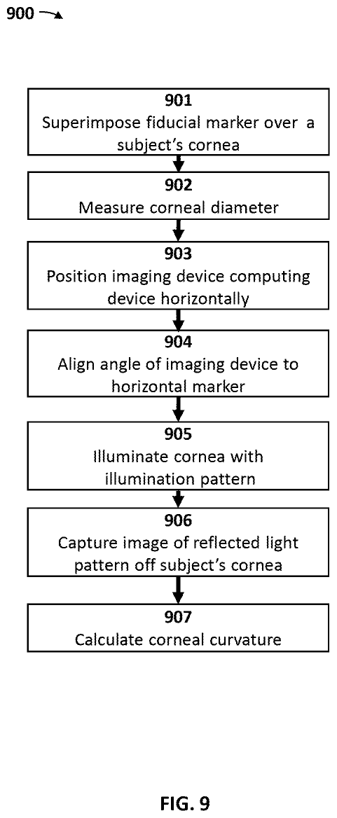

The methods described herein, in some embodiments, may comprise a method for using the corneal topography measurement device to measure a subject's corneal diameter and topography 900, as can be seen in FIG. 9, comprising: superimposing a fiducial marker over a subject's cornea 901; measuring a subject's corneal diameter 902; positioning the imaging device (e.g., mobile computing device) horizontally 903; aligning an angle of the imaging device to a horizontal marker 904; illuminating the subject's cornea with a light pattern 905; capturing an image of the reflected light pattern from the subject's corneal 906; and calculating the subject's corneal curvature and topography 907. In some embodiments, a continual video of a corneal reflected light pattern may be acquired in place of discrete images.

In some embodiments, the methods described herein, may comprise a method for segmenting and/or determining the location of the reflected light pattern of the depth sensing feature overlaid on the topography and/or curvature sensing features. In some cases, method may comprise detecting a location of a depth sensing feature, described elsewhere herein, by matching one or more image pixel data of a reflected light pattern off a subject's cornea to one or more template image pixel data to identify the location of the depth sensing feature. In some cases, the method may further comprise identifying a length of the depth sensing feature that together with the position of the depth sensing feature with respect to the topography and/or curvature sensing feature may provide a distance between the illumination body and/or the imaging device and a cornea of a subject.

### IV. Graphical User Interface Kits

In some embodiments, the device for corneal topographical measurement may comprise a mobile device application with a graphical user interface (GUI) kit to guide the subject or operator to appropriately measure corneal topography.

In some embodiments, the GUI may comprise a marketplace where subjects may purchase contact lenses that are recommended based on the measured corneal diameter and corneal topography. In some embodiments, the marketplace may comprise a series of different windows with contact lens offerings from a plurality of vendors. In some embodiments, the marketplace GUI may comprise a set of customer's provided reviews and rating for the various contact lens offerings by a plurality of vendors

Corneal topographic measurements can provide a rich dataset of diagnostic and prognostic factors that could greatly impact a subject's quality of life. For example, a subject interested in obtaining contact lenses, may have their corneal curvature measured by keratometry to ensure proper fit and function of the contact lens. Without proper measurement of a subject's cornea and fit of a contact lens, the subject may exhibit blurry vision or pain and potential damage to the subject's eye. Corneal topographic measurements may be used as a preventive diagnostic screening tool or to monitor corneal surgical outcomes. To accurately and reproducibly measure the curvature of a subject's cornea, a robust and uniform light source is required. In some cases, the devices and systems of the disclosure provided herein may comprise a device configured to provide a uniform illumination.

In some cases, a uniform illumination source is critical when measuring the contour or topography of a surface. To appropriately measure surface contour or topography of the surface reproducibly and accurately, a structured light pattern should provide a constant spatial intensity i.e., uniform illumination. The uniformity of an illumination source may be determined by a ratio of the brightest region of intensity in a given field of the illumination source to the darkest region of a given filed of the illumination source. A ratio for a given illumination source that approaches a value of 1, indicates a uniform illumination source. The spatial distribution of light intensity of an illumination source may be used to describe a uniform illumination source. In some cases, the spatial gradient of the intensity generated by the illumination source in a field away from the illumination source may be utilized to describe the uniformity of an illumination source. In some instances, a uniform illumination source may comprise a spatial gradient change of up to about 30%, 20%, 10%, 5%, 1%, or less. A uniform illumination source with such a spatial intensity profile may increase the accuracy of an image or data processing method that determines a contour or topography of the surface by measuring a reflected light or light pattern from the surface. Such improvements in accuracy improve the reliability of such a processing method.

In some instances, the device may be configured to couple with a pre-existing light source. In some cases, the light source may be the light source of a smart-phone device. In some instances, the light source may be an independent light source. In some instances, the light source may comprise a light emitting diode (LED), super luminescent diode laser (SLD), laser, or any combination thereof. In some instances, the LED may be configured to output a spectrum of light, e.g., UV, Visible, NIR, or any combination thereof spectrum.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

Any systems, methods, software, compositions, and platforms described herein are modular and not limited to sequential steps. Accordingly, terms such as “first” and “second” do not necessarily imply priority, order of importance, or order of acts.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

1.-51. (canceled)

52. A device for corneal topography measurement, comprising:
an illumination body configured to releasably couple to an imaging device, wherein the illumination body is further configured to (a) project a light pattern onto a cornea of an eye to generate a reflected light pattern and (b) aid transmission of the reflected light pattern from the cornea to a sensor of the imaging device for generating a plurality of light signals, and wherein the projected light pattern comprises illumination of a depth sensing feature configured to generate a depth sensing light pattern of the projected light pattern that provides a distance of the illumination body from a surface of the cornea of the eye.

53. The device of claim 52, wherein the projected light pattern comprises illumination generated by a topography or curvature sensing feature of the illumination body configured to generate a plurality of light signals to determine a topography of the cornea.

54. The device of claim 52, wherein the depth sensing feature comprises a linear or circular feature.

55. The device of claim 52, wherein the depth sensing feature comprises a first region configured to transmit illumination and a second region configured to inhibit illumination.

56. The device of claim 52, wherein the depth sensing feature comprises one or more depth sensing features.

57. The device of claim 52, wherein an optical axis of the sensor of the imaging device is offset from the projected light pattern emitted by the illumination body.

58. The device of claim 52, wherein the projected light pattern is generated by illuminating the illumination body with one or more light sources of the imaging device.

59. The device of claim 58, wherein the one or more light sources of the imaging device are optically coupled to the illumination body by one or more light coupling features of the illumination body.

60. The device of claim 59, wherein the one or more light coupling features of the illumination body comprise a prismatic cavity.

61. A system comprising:
the device of claim 52; and
one or more processors configured to process the plurality of light signals by (i) comparing the projected light pattern emitted by the illumination body to the reflected light pattern off of the cornea to produce a two-dimensional elevation gradient, and (ii) using the two-dimensional elevation gradient to generate a three-dimensional topographic map of the cornea.

62. A method of measuring corneal topography, comprising:
(a) coupling an illumination body to an imaging device, wherein the illumination body comprises a depth sensing feature configured to provide a light pattern to a surface of a cornea that determines a distance between the illumination body and the cornea;
(b) placing the illumination body coupled to the mobile device in proximity to an eye of a subject;
(c) transmitting light from a light source of the imaging device to the illumination body to generate the light pattern on the cornea, wherein the light source of the imaging device is optically coupled to the illumination body that provides the light pattern to the cornea;
(d) using a sensor of the imaging device to receive reflected light of the light pattern off of the cornea to generate a plurality of light signals; and
(e) generating a topography map of the cornea based at least in part on the plurality of light signals.

63. The method of claim 62, wherein the illumination body comprises a topography or curvature sensing feature configured to generate a light pattern that together with the depth sensing feature light pattern is used to generate the topography map of the cornea.

64. The method of claim 63, wherein the illumination body comprises a plurality of lines configured to provide the topography or the curvature sensing feature light pattern.

65. The method of claim 64, wherein the plurality of lines comprises one or more concentric rings.

66. The method of claim 64, wherein the plurality of lines of illumination body is linear.

67. The method of claim 64, wherein the plurality of lines on the illumination body is circular or radial.

68. The method of claim 62, wherein the imaging device comprises a mobile computing device.

69. The method of claim 62, wherein the light source of the image device comprises a light emitting diode.

70. The method of claim 62, wherein the light source comprises one or more light sources of the imaging device.

71. The method of claim 62, wherein the light of the light source is optically coupled to the illumination body by one or more light coupling features of the illumination body.
